SEQUENCE LISTING (17 sequences)

SEQ ID NO: 1 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKHPNGRDTPALFNFTTQELSSNPPLATILIPPHARIQAAASTPTNATAASDANTGDRGQTNNAASASASNST

SEQ ID NO: 2 - DNA, 1260 bp, Homo sapiens
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaacatccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacc

SEQ ID NO: 3 - protein, 737 aa, Homo sapiens
MPLNRTLSMSSLPGLEDWEDEFDLENAVLFEVAWEVANKVGGIYTVLQTKAKVTGDEWGDNYFLVGPYTEQGVRTQVELLEAPTPALKRTLDSMNSKGCKVYFGRWLIEGGPLVVLLDVGASAWALERWKGELWDICNIGVPWYDREANDAVLFGFLTTWFLGEFLAQSEEKPHVVAHFHEWLAGVGLCLCRARRLPVATIFTTHATLLGRYLCAGAVDFYNNLENFNVDKEAGERQIYHRYCMERAAAHCAHVFTTVSQITAIEAQHLLKRKPDIVTPNGLNVKKFSAMHEFQNLHAQSKARIQEFVRGHFYGHLDFNLDKTLYFFIAGRYEFSNKGADVFLEALARLNYLLRVNGSEQTVVAFFIMPARTNNFNVETLKGQAVRKQLWDTANTVKEKFGRKLYESLLVGSLPDMNKMLDKEDFTMMKRAIFATQRQSFPPVCTHNMLDDSSDPILTTIRRIGLFNSSADRVKVIFHPEFLSSTSPLLPVDYEEFVRGCHLGVFPSYYEPWGYTPAECTVMGIPSISTNLSGFGCFMEEHIADPSAYGIYILDRRFRSLDDSCSQLTSFLYSFCQQSRRQRIIQRNRTERLSDLLDWKYLGRYYMSARHMALSKAFPEHFTYEPNEADAAQGYRYPRPASVPPSPSLSRHSSPHQSEDEEDPRNGPLEEDGERYDEDEEAAKDRRNIRAPEWPRRASCTSSTSGRKRNSVDTATSSSLSTPSEPLSPTSSLGEERN

SEQ ID NO: 4 - DNA, 2211 bp, Homo sapiens
atgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtcaggacccaggtggaactgctggaggcccccaccccggccctgaagaggacactggattccatgaacagcaagggctgcaaggtgtatttcgggcgctggctgatcgagggaggccctctggtggtgctcctggacgtgggtgcctcagcttgggccctggagcgctggaagggagagctctgggatatctgcaacatcggagtgccgtggtacgaccgcgaggccaacgacgctgtcctctttggctttctgaccacctggttcctgggtgagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtctttctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggccgcaagcgcaactctgtggacacggccacctccagctcactcagcaccccgagcgagcccctcagccccaccagctccctgggcgaggagcgtaac

SEQ ID NO: 5 - protein, 26 aa, Artificial Sequence
  Description of Artificial Sequence Synthetic PRT
YRRAAVPPSPSLSRHSSPHQSEDEEE

SEQ ID NO: 6 - DNA, 24 bp, Artificial Sequence
  Description of Artificial Sequence synthetic DNA
agggtatgataaccgggagatcgt

SEQ ID NO: 7 - DNA, 24 bp, Artificial Sequence
  Description of Artificial Sequence synthetic DNA
gggccatatagttccacaaaggca

SEQ ID NO: 8 - DNA, 24 bp, Artificial Sequence
  Description of Artificial Sequence synthetic DNA
caaaaggcactggaactcgcaatg

SEQ ID NO: 9 - DNA, 24 bp, Artificial Sequence
  Description of Artificial Sequencesynthetic DNA
ttcttggcaacggcaacaaaccac

SEQ ID NO: 10 - DNA, 24 bp, Artificial Sequence
  Description of Artificial Sequence synthetic DNA
ggtgaatcgagaagagccatcatg

SEQ ID NO: 11 - DNA, 24 bp, Artificial Sequence
  Description of Artificial Sequence synthetic DNA
ttcaggtagagttggaggctgatg

SEQ ID NO: 12 - DNA, 25 bp, Artificial Sequence
  Description of Artificial Sequence synthetic DNA
gaagaactttcctgatggccaccag

SEQ ID NO: 13 - DNA, 25 bp, Artificial Sequence
  Description of Artificial Sequence synthetic DNA
ctggtggccatcaggaaagttcttc

SEQ ID NO: 14 - RNA, 21 nt, Artificial Sequence
  Description of Artificial Sequence synthetic RNA
gucaguuacacagacacuauu

SEQ ID NO: 15 - RNA, 21 nt, Artificial Sequence
  Description of Artificial Sequence synthetic RNA
uagugucuguguaacugacuu

SEQ ID NO: 16 - RNA, 21 nt, Artificial Sequence
  Description of Artificial Sequence synthetic RNA
gucuagccuauauccauucuu

SEQ ID NO: 17 - RNA, 21 nt, Artificial Sequence
  Description of Artificial Sequence synthetic RNA
gaauggauauaggcuagacuu

(19) United States
(12) Patent Application Publication
(10) Pub. No.: US 2006/0217368 A1
(43) Pub. Date: Sep. 28, 2006

(54) Drug for nerve regeneration

(75) Inventors: Tsuyoshi Morishita, Tokyo (JP); Kazuhiro Sakurada, Kanagawa (JP); Keiko Suzuki, Tokyo (JP); Shun-ichi Ikeda, Tokyo (JP)

Correspondence Address: FITZPATRICK CELLA HARPER & SCINTO, 30 ROCKEFELLER PLAZA, NEW YORK, NY, 10112, US

(73) Assignee: Kyowa Hakko Kogyo Co., Ltd., Chiyoda-ku (JP)

(21) Appl. No.: 10/552,061

(22) PCT Filed: Apr. 16, 2004
(86) PCT No.: PCT/JP04/05503

§ 371 Date: Oct. 4, 2005

(30) Foreign Application Priority Data
Apr. 18, 2003 (JP) 2003114579

(51) Int. Cl. A61K 31/55; A61K 31/404; A61K 33/00; A61K 31/407; A61K 31/405

(52) U.S. Cl. 514/212.06; 514/414; 424/722; 514/410

(57) ABSTRACT

An object of the present invention is to provide a nerve regenerating drug, an agent for the promotion of neuropoiesis of a neural stem cell, a neuron obtained by culturing a neural stem cell in the presence of the agent for the promotion of neuropoiesis, and a method of the manufacture of the neuron. In order to achieve the object, the invention provides a nerve regenerating drug comprising a substance that inhibits the activity of glycogen synthase kinase-3, as an active ingredient; an agent for the promotion of neuropoiesis of a neural stem cell comprising the substance as an active ingredient; a neuron obtained by culturing a neural stem cell in the presence of the agent for the promotion of neuropoiesis; and a method of the manufacture of the neuron. The medical drug according to the invention is useful as a therapeutic drug for neurological diseases such as Parkinson's disease, Alzheimer's disease, Down's disease, cerebrovascular disorder, cerebral stroke, spinal cord injury, Huntington's chorea, multiple sclerosis, amyotrophic lateral sclerosis, epilepsy, anxiety disorder, schizophrenia, depression and manic depressive psychosis.

### TECHNICAL FIELD

The present invention relates to a nerve regenerating drug comprising a substance that inhibits the activity of glycogen synthase kinase-3 (hereinafter, abbreviated as GSK-3), as an active ingredient; an agent for the promotion of neuropoiesis comprising a substance that inhibits the activity of GSK-3, as an active ingredient; a neuron obtained by culturing a neural stem cell in the presence of the agent for the promotion of neuropoiesis; and a method of the manufacture of the neuron.

### BACKGROUND ART

Neurological diseases collectively refer to neurodegenerative diseases in which a brain or a peripheral neuron is injured due to a genetic factor, an environmental factor, an aging factor or the like; depression and manic depressive psychos is not accompanied by degeneration of a nerve; and the like. Specific examples of the neurodegenerative disease include Parkinson's disease, Alzheimer's disease, polyglutamic acid disease, amyotrophic lateral sclerosis, polyneuropathy, spinal cord injury, cerebrovascular disorder and the like. Although general therapeutic methods of these neurodegenerative diseases are therapies in which a neurotransmitter which was lost due to the injury of a neuron is supplied, diseases of which symptoms may be ameliorated by the therapy are limited to Parkinson's disease, Alzheimer's disease and the like. Further, progress of the nerve cell death can not be interrupted by the supplementary therapy of a neurotransmitter.

Although regenerative medical treatments for regenerating a central nervous system have been studied as a therapeutic method for positively recovering the functions of a dopaminergic neuron which was lost in Parkinson's disease, they involve a variety of problems, because they are methods in which the brain of an aborted fetus is used. Therefore, they have not been applied for general utilization.

Moreover, a therapeutic method in which neural stem cells obtained from the brain of an aborted fetus or ES cells obtained from a human fertilized egg are subjected to large scale culture followed by use in transplantation after converting into an intended neuron has also been studied, however, a technique for allowing accurate differentiation into the intended neuron has not been established, and is also involved problems resulting from the method in which neural stem cells derived from a fetus or human ES cells are used. Thus, clinical applications have not been advanced.

On the other hand, a therapeutic method of neurodegenerative diseases in which neural stem cells endogenously existing in the brain of a patient are stimulated by an agent or the like to induce regeneration has been studied on the basis of reports on separation of neural stem cells from an adult brain, suggesting the occurrence of neogenesis of a neuron also in a human adult brain during a lifetime.

Promotion of neuropoiesis in hippocampus or olfactory bulb by an intracranial administration of a cytokine or by a disease model has been reported such as: insulin like growth factor-1 [J. Neuroscience, 20, 2896-2903 (2000)], a fibroblast growth factor-2 [Pro. Nat. Acad. Sci. USA, 98, 5874-5879 (2001)], a stem cell factor [J. Clin. Invest., 110, 311-319 (2002)], erythropoietin [J. Neuroscience, 21, 9733-9743 (2001)], whole brain ischaemia [J. Neuroscience, 18, 7768-7778 (1998)] and stimulus epilepticus [J. Neuroscience, 22, 3174-3188 (2002)]. Further, it is also reported that neogenesis of a dopaminergic neuron occurs in corpus striatum by intracranially administering a tumor growth factor-α to ameliorate the symptom of Parkinson's disease [Pro. Nat. Acad. Sci. USA, 97, 14686-14691 (2000)]. Additionally, it is also reported that 40% of CA1 pyramidal cells lost due to ischemic injury in hippocampus are completely recovered by intracranially administering a fibroblast growth factor-2 and an epidermal growth factor on day 2 to day 5 post the ischaemia [Cell, 110, 429-441 (2002)].

However, any one of the aforementioned methods requires intracerebral administration of a proteinous factor, therefore, applications to general medical treatments have been difficult.

Examples of reported low molecular weight compound that can be peripherally administered and can result in neuropoiesis include antidepressants such as monoamine oxidase inhibitors, serotonin specific transporter inhibitors and phosphodiesterase IV inhibitors [Neuropsychopharmacology, 25, 836-844 (2001)]. As mechanisms which may involve intracerebral induction of nerve regeneration by these agents, it has been believed that the agent promotes neuropoiesis around a serotonergic neuron through directly or indirectly acting on a serotonin receptor signal of the serotonergic neuron to produce a neurotrophic factor. Therefore, it is believed that these agents can not be utilized as a nerve regenerating drug for almost all neurological diseases that are not related to degeneration of the serotonergic neuron.

Further, it is reported that lithium that is a mood stabilizing drug protects a neogenetic neuron, which is newly and constitutively generated in hippocampus, from death of the neuron through inducing the expression of a cell death suppressive gene bcl-2, thereby apparently increasing neuropoiesis in hippocampus, [J. Neurochemistry, 75, 1729-1734 (2000)]. In addition, lithium is also reported to induce the expression of a neurotrophic factor BDNF [Neuropharmacology, 43, 1173-1179 (2002)]. However, it has not been reported that lithium promotes neuropoiesis by directly acting on a neural stem cell and promoting neuronal differentiation, and also, the activity of lithium to promote neuropoiesis in a region other than hippocampus has not been reported. Furthermore, it has not been known as to why lithium has a therapeutic effect on depression and manic depressive psychosis not accompanied by degeneration of a nerve.

In connection with Alzheimer's disease, a hypothesis was proposed stating that glycogen synthase kinase-3 (hereinafter, abbreviated as GSK-3) excessively phosphorylates a tau protein that is a microtubule-associated protein thereby resulting in neurofibrillary alteration to induce nerve cell death [Trends in Molecular Medicine, 8,126-132 (2002)]. Further, a substance that inhibits the activity of GSK-3 was reported to have an activity to protect mature nerve cells in vitro [J. Neurochemistry, 77, 94-102 (2001)]. On the basis of this report, it has been believed that the substance that inhibits the activity of GSK-3 can be used as a therapeutic drug for various neurodegenerative diseases in addition to Alzheimer's disease (Pamphlet of International Patent Publication No. 00/38675), however, it has not been known whether or not a neurodegenerative disease can be actually treated by protecting a mature neuron, and that there exists a neuropoiesis promoting action of the substance that inhibits the activity of GSK-3.

### DISCLOSURE OF THE INVENTION

An object of the present invention is to provide a nerve regenerating drug comprising a substance that inhibits the activity of GSK-3, as an active ingredient; an agent for the promotion of neuropoiesis of a neural stem cell comprising the substance as an active ingredient; a neuron obtained by culturing a neural stem cell in the presence of the agent for the promotion of neuropoiesis; and a method of the manufacture of the neuron.

The present invention relates to the following items (1) to (41).

(1) A nerve regenerating drug which comprises a substance that inhibits the activity of glycogen synthase kinase-3 (hereinafter, abbreviated as GSK-3), as an active ingredient.

(2) The medical drug according to the above item (1) wherein the nerve regenerating drug is a therapeutic drug for a neurological disease.

(3) The medical drug according to the above item (2) wherein the neurological disease is selected from the group consisting of Parkinson's disease, Alzheimer's disease, Down's disease, cerebrovascular disorder, cerebral stroke, spinal cord injury, Huntington's chorea, multiple sclerosis, amyotrophic lateral sclerosis, epilepsy, anxiety disorder, schizophrenia, depression and manic depressive psychosis.

(4) The medical drug according to any one of the above items (1) to (3) wherein the substance that inhibits the activity of GSK-3 is lithium or a pharmacologically acceptable salt thereof.

(5) The medical drug according to any one of the above items (1) to (3) wherein the substance that inhibits the activity of GSK-3 is a bisindolylmaleimide derivative, a 3-aryl-4-indolylmaleimide derivative, an indolocarbazole derivative, an indolo[3,2-d][1]benzazepin-6(5H)-one derivative or an indirubin derivative, or a pharmacologically acceptable salt thereof.

(6) The medical drug according to any one of the above items (1) to (3) wherein the substance that inhibits the activity of GSK-3 is a compound represented by the formula (I): 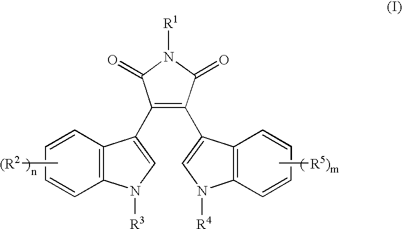
[wherein n and m may be the same or different, and represent an integer of 1 to 3; R¹, R³ and R⁴ may be the same or different, and represent hydrogen, substituted or unsubstituted lower alkyl, substituted or unsubstituted lower alkenyl, —COR⁶ (wherein R⁶ represents hydrogen, substituted or unsubstituted lower alkyl, substituted or unsubstituted lower alkenyl, substituted or unsubstituted aryl or substituted or unsubstituted cycloalkyl), —COOR⁷ (wherein R⁷ represents hydrogen, substituted or unsubstituted lower alkyl, substituted or unsubstituted aryl or substituted or unsubstituted cycloalkyl) or —OR⁸ (wherein R⁸ represents hydrogen, substituted or unsubstituted lower alkyl, substituted or unsubstituted aryl or substituted or unsubstituted cycloalkyl); R² and R⁵ may be the same or different, and represent hydrogen, substituted or unsubstituted lower alkyl, a substituted or unsubstituted lower alkenyl, substituted or unsubstituted lower alkoxy, substituted or unsubstituted lower alkoxycarbonyl, substituted or unsubstituted aryl, carboxy, halogen, hydroxy, nitro, amino, or mono- or di-lower alkylamino; when n and m are 2 or 3, each of R² and R⁵ may be the same or different], a compound represented by the formula (II): 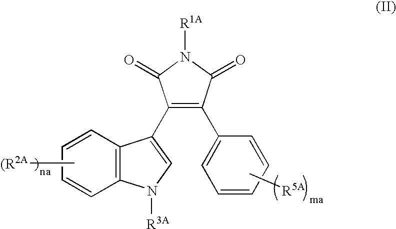
(wherein na, ma, R^{1A}, R^{2A}, R^{3A} and R^{5A} are as defined for the aforementioned n, m, R¹, R², R³ and R⁵, respectively) or a compound represented by the formula (III): 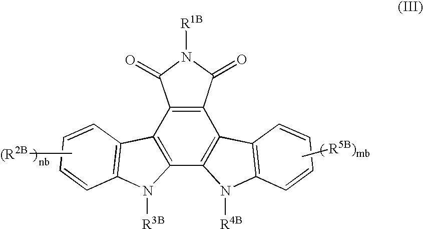
[wherein nb, mb, R^{1B}, R^{2B} and R^{5B} are as defined for the aforementioned n, m, R¹, R² and R⁵, respectively; R^{3B} and R^{4B} may be the same or different, and represent hydrogen, substituted or unsubstituted lower alkyl, substituted or unsubstituted lower alkenyl, —COR⁶ (wherein R⁶ is as defined above), —COOR⁷ (wherein R⁷ is as defined above) or —OR⁸ (wherein R⁸ is as defined above), or R^{3B} and R^{4B} together form 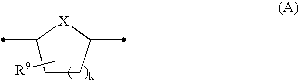
(wherein k represents 1 or 2; X represents CH₂, NH, an oxygen atom or a sulfur atom; R⁹ represents hydroxy, carboxy, carbamoyl or lower alkoxycarbonyl)], or a pharmacologically acceptable salt thereof.

(7) The medical drug according to any one of the above items (1) to (3) wherein the substance that inhibits the activity of GSK-3 is a compound represented by the formula (Ia): 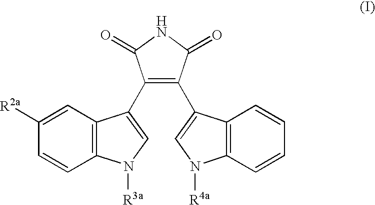
(wherein R^{2a} represents hydrogen, lower alkoxy, lower alkoxycarbonyl, aryl or nitro; R^{3a} and R^{4a} may be the same or different, and represent substituted or unsubstituted lower alkyl), or a pharmacologically acceptable salt thereof.

(8) The medical drug according to any one of the above items (1) to (3) wherein the substance that inhibits the activity of GSK-3 is a compound represented by the formula (IIa): 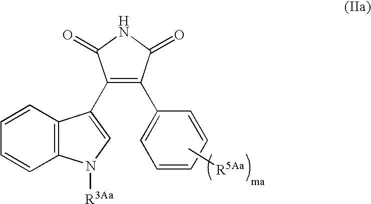
(wherein ma is as defined above; R^{3Aa} represents substituted or unsubstituted lower alkyl; R^{5Aa} represents halogen), or a pharmacologically acceptable salt thereof.

(9) The medical drug according to any one of the above items (1) to (3) wherein the substance that inhibits the activity of GSK-3 is a compound represented by the formula (IIIa): 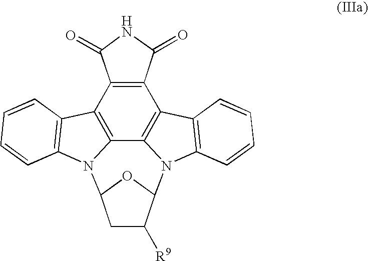
(wherein R⁹ is as defined above), or a pharmacologically acceptable salt thereof.

(10) The medical drug according to any one of the above items (1) to (3) wherein the substance that inhibits the activity of GSK-3 is a compound selected from the group consisting of 3,4-bis(1-methylindole-3-yl)-1H-pyrrole-2,5-dione, 3-(1-methylindole-3-yl)-4-(1-propylindole-3-yl)-1H-pyrrole-2,5-dione, 3-[1-(3-cyanopropyl)indole-3-yl]-4-(1-methyl-indole-3-yl)-1H-pyrrole-2,5-dione, 3-[1-(3-aminopropyl)-indole-3-yl]-4-(1-methylindole-3-yl)-1H-pyrrole-2,5-dione, 3-[1-(3-carboxypropyl)indole-3-yl]-4-(1-methyl-indole-3-yl)-1H-pyrrole-2,5-dione, 3-[1-(3-carbamoyl-propyl)indole-3-yl]-4-(1-methylindole-3-yl)-1H-pyrrole-2,5-dione, 3-[1-(3-aminopropyl)indole-3-yl]-4-(1-methyl-5-propyloxyindole-3-yl)-1H-pyrrole-2,5-dione, 3-[1-(3-hydroxypropyl)indole-3-yl]-4-(1-methyl-5-phenylindole-3-yl)-1H-pyrrole-2,5-dione, 3-[1-(3-aminopropyl)indole-3-yl]-4-(1-methyl-5-phenylindole-3-yl)-1H-pyrrole-2,5-dione, 3-[1-(3-hydroxypropyl)indole-3-yl]-4-(1-methyl-5-methoxycarbonylindole-3-yl)-1H-pyrrole-2,5-dione, 3-[1-(3-hydroxypropyl)indole-3-yl]-4-(1-methyl-5-nitroindole-3-yl)-1H-pyrrole-2,5-dione, 3-(1-methylindole-3-yl)-4-[1-(3-hydroxypropyl)-5-nitroindole-3-yl]-1H-pyrrole-2,5-dione, 3-(2-chlorophenyl)-4-(1-methylindole-3-yl)-1H-pyrrole-2,5-dione, 3-(2,4-dichlorophenyl)-4-(1-methylindole-3-yl)-1H-pyrrole-2,5-dione, 3-(2-chlorophenyl)-4-[1-(3-hydroxypropyl)indole-3-yl]-1H-pyrrole-2,5-dione, 4-[1-(3-aminopropyl)indole-3-yl]-3-(2-chlorophenyl)-1H-pyrrole-2,5-dione and 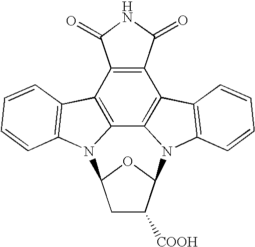
or a pharmacologically acceptable salt thereof.

(11) The medical drug according to any one of the above items (1) to (3) wherein the substance that inhibits GSK-3 is a compound represented by the formula (IV): 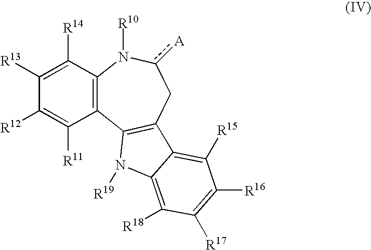
[wherein A is oxygen or sulfur coupled to the right by a single or double bond; R¹⁰ is selected from the group consisting of hydrogen, aryl, lower aliphatic substituents, particularly alkyl and lower alkyl ester; R¹¹-R¹⁴ are independently selected from the group consisting of alkoxy, amino, acyl, aliphatic substituents, particularly alkyl, alkenyl and alkinyl substituents, aliphatic alcohols, particularly alkyl alcohols, aliphatic nitriles, particularly alkyl nitriles, cyano, nitro, carboxyl, halogen, hydrogen, hydroxyl, imino and α,β-unsaturated ketones; R¹⁵-R¹⁸ are independently selected from the group consisting of aliphatic substituents, particularly alkyl, alkenyl and alkinyl substituents, particularly lower aliphatic substituents, alipahatic alcohols, particularly alkyl alcohols, alkoxy, acyl, cyano, nitro, epoxy, haloalkyl groups, halogen, hydrogen and hydroxyl; R¹⁹ is selected from the group consisting of aliphatic groups, particularly lower alkyl groups, aliphatic alcohols, particularly alkyl alcohols, carboxylic acids and hydrogen], or a pharmacologically acceptable salt thereof.

(12) The medical drug according to any one of the above items (1) to (3) wherein the substance that inhibits GSK-3 is a compound selected from the group consisting of 7,12-dihydro-indolo[3,2-d][1]benzazepin-6(5H)-one, 2-bromo-7,12-dihydro-indolo[3,2-d][1]benzazepin-6(5H)-one, 9-bromo-7,12-dihydro-indolo[3,2-d][1]benzazepin-6(5H)-one, 9-chloro-7,12-dihydro-indolo[3,2-d][1]benzazepin-6(5H)-one, 11-chloro-7,12-dihydro-indolo[3,2-d][1]benzazepin-6(5H)-one, 10-bromo-7,12-dihydro-indolo[3,2-d][1]benzazepin-6(5H)-one, 8-bromo-6,11-dihydro-thieno[3′,2′:2,3]azepino-[4,5-b]indol-5(4H)-one, 9-bromo-7,12-dihydro-4-methoxy-indolo[3,2-d][1]benzazepin-6(5H)-one, 9-bromo-7,12-dihydro-4-hydroxy-indolo[3,2-d][1]benzazepin-6(5H)-one, 7,12-dihydro-4-methoxy-indolo[3,2-d][1]benzazepin-6(5H)-one, 9-bromo-7,12-dihydro-2,3-dimethoxy-indolo[3,2-d][1]benzazepin-6(5H)-one, 9-bromo-7,12-dihydro-2,3-di-hydroxy-indolo[3,2-d][1]benzazepin-6(5H)-one, 7,12-dihydro-2,3-dimethoxy-indolo[3,2-d][1]benzazepin-6(5H)-one, 7,12-dihydro-9-trifluormethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2-bromo-7,12-dihydro-9-trifluoromethyl-indolo[3,2-d][1]benzazepin-6(5H)-one, 9-bromo-7,12-dihydro-indolo[3,2-d][1]benzazepin-6(5H)-thione, 9-bromo-5,12-bis-(t-butyloxycarbonyl)-7,12-dihydro-indolo[3,2-d][1]benzazepin-6(5H)-one, 9-bromo-12-(t-butyloxycarbonyl)-7,12-dihydro-indolo[3,2-d][1]benzazepin-6(5H)-one, 9-bromo-5,7-bis-(t-butyloxycarbonyl)-7,12-dihydro-indolo[3,2-d][1]benzazepin-6(5H)-one, 9-bromo-5,7,12-tri-(t-butyloxycarbonyl)-7,12-dihydro-indol o[3,2-d][1]benzazepin-6(5H)-one, 9-bromo-7,12-dihydro-5-methyloxycarbonylmethyl-indolo[3,2-d][1]benzazepin-6(5H)-one, 9-bromo-7,12-dihydro-12-methyloxycarbonylmethyl-indolo[3,2-d][1]benzazepin-6(5H)-one, 9-bromo-7,12-dihydro-12-(2-hydroxyethyl)-indolo[3,2-d][1]benzazepin-6-(5H)-one, 2,9-dibromo-7,12-dihydro-indolo[3,2-d][1]benzazepin-6(5H)-one, 8,10-dichloro-7,12-dihydro-indolo-[3,2-d][1]benzazepin-6(5H)-one, 9-cyano-7,12-dihydro-indolo[3,2-d][1]benzazepin-6(5H)-one, 9-bromo-7,12-dihydro-5-methyl-indolo[3,2-d][1]benzazepin-6(5H)-one, 5-benzyl-9-bromo-7,12-dihydro-5-methyl-indolo[3,2-d][1]-benzazepin-6(5H)-one, 9-bromo-7,12-dihydro-12-methyl-indolo-[3,2-d][1]benzazepin-6(5H)-one, 9-bromo-12-ethyl-7,12-dihydro-indolo[3,2-d][1]benzazepin-6(5H)-one, 9-bromo-7,12-dihydro-12-(2-propenyl)-indolo[3,2-d][1]benzazepin-6(5H)-one, 7,12-dihydro-9-methyl-indolo[3,2-d][1]-benzazepin-6(5H)-one, 7,12-dihydro-9-methoxy-indolo[3,2-d][1]benzazepin-6(5H)-one, 9-fluoro-7,12-dihydro-12-(2-propenyl)-indolo[3,2-d][1]benzazepin-6(5H)-one, 11-bromo-7,12-dihydro-indolo[3,2-d][1]benzazepin-6(5H)-one, 9-bromo-7,12-dihydro-2-(methyliminoamine)-indolo[3,2-d][1]benzazepin-6(5H)-one, 9-bromo-7,12-dihydro-2-(carboxylic acid)-indolo[3,2-d][1]benzazepin-6(5H)-one, 9-bromo-7,12-dihydro-10-hydroxy-indolo[3,2-d][1]benzazepin-6(5H)-one, 9-bromo-7,12-dihydro-11-hydroxymethyl-indolo[3,2-d][1]-benzazepin-6(5H)-one, 7,12-dihydro-4-hydroxy-indolo[3,2-d][1]benzazepin-6(5H)-one 7,12-dihydro-2,3-dihydroxy-indolo[3,2-d][1]benzazepin-6(5H)-one, 2,3-dimethoxy-9-nitro-7,12-dihydro-indolo[3,2-d][1]benzazepin-6(5H)-one, 9-cyano-7,12-dihydro-indolo[3,2-d][1]benzazepin-6(5H)-one, 2,3-dimethoxy-9-cyano-7,12-dihydro-indolo[3,2-d][1]benzazepin-6(5H)-one, 9-nitro-7,12-dihydro-indolo[3,2-d][1]benzazepin-6(5H)-one, 3-(6-oxo-9-trifluoromethyl-5,6,7,12-tetrahydro-indolo[3,2-d][1]benzazepin-2-yl)-propionitrile, 2-bromo-9-nitro-7,12-dihydro-indolo[3,2-d][1]benzazepin-6(5H)-one, 3-(6-oxo-9-trifluoromethyl-5,6,7,12-tetrahydro-indolo[3,2-d][1]benzazepin-2-yl)acrylonitrile, 2-(3-hydroxy-1-propinyl)-9-trifluoromethyl-7,12-dihydro-indolo[3,2-d][1]benzazepin-6(5H)-one, 2-iodo-9-bromo-7,12-dihydro-indolo[3,2-d][1]benzazepin-6(5H)-one, 2-(3-oxo-1-butenyl)-9-trifluoromethyl-7,12-tetrahydro-indolo[3,2-d][1]benzazepin-6(5H)-one, 8-chloro-6,11-dihydro-thieno[3′,2′:2,3]azepino[4,5-b]indol-5(4H)-one, 2-iodo-9-trifluoromethyl-7,12-dihydro-indolo[3,2-d][1]-benzazepin-6(5H)-one, 7,12-dihydro-pyrido[3′,2′:4,5]-pyrrolo[3,2-d][1]benzazepin-6(5H)-one, 11-methyl-7,12-dihydro-indolo[3,2-d][1]-benzazepin-6(5H)-one, 2-[2-(1-hydroxycyclohexyl)-ethinyl]-9-trifluoromethyl-7,12-dihydro-indolo-[3,2-d][1]benzazepin-6(5H)-one, 2-cyano-7,12-dihydro-indolo[3,2-d][1]benzazepin-6(5H)-one, 2-iodo-7,12-dihydro-indolo[3,2-d][1]benzazepin-6(5H)-one, 11-ethyl-7,12-dihydro-indolo[3,2-d][1]benzazepin-6(5H)-one, 8-methyl-6,11-dihydro-thieno[3′,2′:2,3]azepino[4,5-b]indol-5(4H)-one and 3-(6-oxo-9-trifluoromethyl-5,6,7,12-tetrahydro-indolo[3,2-d][1]benzazepin-2-yl)acrylic acid methyl ester, or a pharmacologically acceptable salt thereof.

(13) The medical drug according to any one of the above items (1) to (3) wherein the substance that inhibits GSK-3 is a compound selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]-benzazepin-6(5H)-one, 7,12-dihydro-9-trifluoromethyl-indolo-[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indole-5(4H)-one, 7,12-dihydro-9-methoxy-indolo[3,2-d][1]benzazepin-6(5H)-one, 10-bromo-7,12-dihydro-indolo[3,2-d][1]benzazepin-6(5H)-one, 11-bromo-7,12-dihydro-indolo[3,2-d][1]-benzazepin-6(5H)-one, 11-chloro-7,12-dihydro-indolo[3,2-d]-[1]benzazepin-6(5H)-one, 9-fluoro-7,12-dihydro-indolo-[3,2-d][1]benzazepin-6(5H)-one, 9-methyl-7,12-dihydro-indolo[3,2-d][1]benzazepin-6(5H)-one, 9-bromo-7,12-dihydro-indolo[3,2-d][1]benzazepin-6(5H)-thione, 8,10-dichloro-7,12-dihydro-indolo[3,2-d][1]benzazepin-6(5H)-one, 9-bromo-7,12-dihydro-12-(2-hydroxyethyl)-indolo[3,2-d][1]-benzazepin-6(5H)-one, 9-bromo-7,12-dihydro-2,3-dihydroxy-indolo[3,2-d][1]benzazepin-6(5H)-one, 2-bromo-7,12-dihydro-indolo[3,2-d][1]benzazepin-6(5H)-one, 7,12-dihydro-2,3-dimethoxy-indolo[3,2-d][1]benzazepin-6(5H)-one, 9-bromo-7,12-dihydro-12-methyl-indolo[3,2-d][1]benzazepin-6(5H)-one, 9-bromo-7,12-dihydro-5-methyloxycarbonylmethyl-indolo[3,2-d][1]benzazepin-6(5H)-one and 7,12-dihydro-indolo[3,2-d][1]benzazepin-6(5H)-one, or a pharmacologically acceptable salt thereof.

(14) The medical drug according to any one of the above items (1) to (3) wherein the substance that inhibits GSK-3 is a compound selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]-benzazepin-6(5H)-one, 7,12-dihydro-9-trifluormethyl-indolo-[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indol-5(4H)-one and 7,12-dihydro-9-methoxy-indolo[3,2-d][1]benzazepin-6(5H)-one, or a pharmacologically acceptable salt thereof.

(15) The medical drug according to any one of the above items (1) to (3) wherein the substance that inhibits GSK-3 is 9-bromo-7,12-dihydro-indolo[3,2-d][1]benzazepin-6(5H)-one or a pharmacologically acceptable salt thereof.

(16) The medical drug according to any one of the above items (1) to (3) wherein the substance that inhibits GSK-3 is a compound represented by the formula (V): 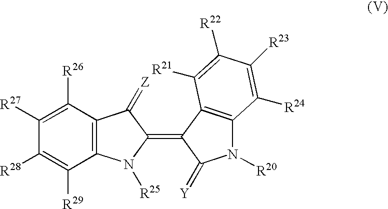
[wherein R²⁰ and R²⁵ which may be the same or different represent hydrogen; halogen; a hydroxy group; a methylene hydroxy group; a straight chain or branched C₁ to C₁₈-alkyl or alkoxy or methylenealkoxy group; a cycloalkyl group having 3 to 7 carbon atoms, and including one or more heteroatoms as needed; a substituted or unsubstituted aryl, aralkyl or aryloxy group having one or more heteroatoms as needed; a mono-, di- or trialkylsilyl group each independently having 1 to 6 carbon atoms within the straight chain or branched alkyl group; a mono-, di- or triarylsilyl group each independently having a substituted or unsubstituted aryl group; a trifluoromethyl group; —COM; —COOM; or a —CH₂COOM group (wherein M represents hydrogen, a straight chain or branched C₁ to C₁₈-alkyl group substituted with one or more hydroxy and/or amino groups if necessary, or an aryl group, which may be substituted with one or more halogen, alkyl groups or alkoxy groups, having one or more heteroatoms if necessary); an —NR³⁰R³¹ group (wherein R³⁰ and R³¹ which may be the same or different represent hydrogen, a C₁, to C₁₈ straight chain or branched alkyl group additionally substituted with one or more hydroxy and/or amino groups if necessary, a substituted or unsubstituted aryl group including one or more heteroatoms if necessary); an acyl group; a —CH₂—NR³⁰R³¹ methyleneamino group (wherein R³⁰ and R³¹ have the meanings as defined above); a benzyl group having one or more heteroatoms in the benzene ring if necessary; a methylenecycloalkyl group having 3 to 7 carbon atoms, and including one or more heteroatoms if necessary; a physiological amino acid group coupled to a nitrogen atom as an amide; an O-glycoside or N-glycoside of which glycoside being selected from monosaccharides or disaccharides; or a methylenesulfonate group; R²¹, R²², R²³, R²⁴, R²⁶, R²⁷, R²⁸ and R²⁹ which may be the same or different represent hydrogen; halogen; a hydroxy group; a nitroso group; a nitro group; an alkoxy group; a straight chain or branched C₁ to C₁₈ alkyl group substituted with one or more hydroxy and/or amino groups if necessary; a substituted or unsubstituted aryl group having one or more heteroatoms if necessary; a substituted or unsubstituted aralkyl group having one or more heteroatoms if necessary; a substituted or unsubstituted aryloxy group having one or more heteroatoms if necessary; a substituted or unsubstituted methylenearyloxy group having one or more heteroatoms if necessary; a cycloalkyl group having 3 to 7 carbon atoms, and including one or more heteroatoms if necessary; a methylenecycloalkyl group having 3 to 7 carbon atoms, and including one or more heteroatoms if necessary; a trifluoromethyl group; —COM; —COOM; or a CH₂COOM group (wherein M represents hydrogen, a straight chain or branched C₁ to C₁₈-alkyl group additionally substituted with one or more hydroxy and/or amino groups if necessary, or an aryl group, which may be substituted with one or more halogen atoms, alkyl groups or alkoxy groups, having one or more heteroatoms if necessary); an —NR³⁰R³¹ group (wherein R³⁰ and R³¹ which may be the same or different represent hydrogen, a straight chain or branched C₁, to C₁₈-alkyl group additionally substituted with one or more hydroxy and/or amino groups if necessary, a substituted or unsubstituted aryl group including one or more heteroatoms if necessary, an acyl group; or form a part of cycloalkyl having 3 to 7 carbon atoms with the nitrogen atom including one or more heteroatoms if necessary); a —CONR³⁰R³¹ group (wherein R³⁰ and R³¹ have the meanings as defined above); a hydroxylamino group; a phosphate group; a phosphonate group; a sulfate group; a sulfonate group; a sulfonamide group; an —SO₂NR³⁰R³¹ group (wherein R³⁰ and R³¹ have the meanings as defined above); an —N═N—R³² azo group (wherein R³² represents an aromatic group substituted with one or more carboxyl, phosphoryl or sulfonate groups if necessary, or an O-glycoside or N-glycoside group of which glycoside being selected from monosaccharides or disaccharides); or R²⁰ and R²⁴, and R²⁵ and R²⁹ together form a ring having one to four CH₂ groups each independently substituted if necessary, respectively; Y and Z which may be the same or different represent an oxygen; sulfur; selenium; tellurium atom; an NR³³ group (wherein R³³ represents hydrogen, a straight chain or branched C₁ to C₁₈ alkyl group substituted with one or more carboxyl, phosphoryl or sulfonate groups if necessary, a substituted or unsubstituted aryl group including one or more heteroatoms if necessary, an aralkyl group or a sulfonate group); or —NOR³³ (wherein R³³ group have the meanings as defined above)], or a pharmacologically acceptable salt thereof.

(17) The medical drug according to any one of the above items (1) to (3) wherein the substance that inhibits GSK-3 is a compound selected from the group consisting of indirubin, 5-iodo-indirubin, 5-bromo-indirubin, 5-chloro-indirubin, 5-fluoro-indirubin, 5-methyl-indirubin, 5-nitro-indirubin, 5-SO₃H-indirubin, 5′-bromo-indirubin, 5-5′-dibromo-indirubin and 5′-bromo-indirubin 5-sulfonic acid, or a pharmacologically acceptable salt thereof.

(18) The medical drug according to any one of the above items (1) to (3) wherein the substance that inhibits GSK-3 is a compound selected from the group consisting of indirubin-3′-monooxime, 5-iodo-indirubin-3′-monooxime and 5-SO₃Na-indirubin-3′-monooxime, or a pharmacologically acceptable salt thereof.

(19) The medical drug according to any one of the above items (i) to (3) wherein the substance that inhibits GSK-3 is indirubin-3′-monooxime or a pharmacologically acceptable salt thereof.

(20) An agent for the promotion of neuropoiesis of a neural stem cell which comprises a substance that inhibits the activity of GSK-3, as an active ingredient.

(21) The agent for the promotion of neuropoiesis according to the above item (20) wherein the substance that inhibits the activity of GSK-3 is lithium or a pharmacologically acceptable salt thereof.

(22) The agent for the promotion of neuropoiesis according to the above item (20) wherein the substance that inhibits the activity of GSK-3 is a bisindolylmaleimide derivative, a 3-aryl-4-indolylmaleimide derivative, an indolocarbazole derivative, an indolo[3,2-d][1]benzazepin-6(5H)-one derivative or an indirubin derivative, or a pharmacologically acceptable salt thereof.

(23) The agent for the promotion of neuropoiesis according to the above item (20) wherein the substance that inhibits the activity of GSK-3 is a compound represented by the formula (I): 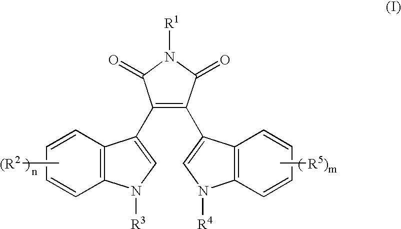
[wherein n and m may be the same or different, and represent an integer of 1 to 3; R¹, R³ and R⁴ may be the same or different, and represent hydrogen, substituted or unsubstituted lower alkyl, substituted or unsubstituted lower alkenyl, —COR⁶ (wherein R⁶ represents hydrogen, substituted or unsubstituted lower alkyl, substituted or unsubstituted lower alkenyl, substituted or unsubstituted aryl or substituted or unsubstituted cycloalkyl), —COOR⁷ (wherein R⁷ represents hydrogen, substituted or unsubstituted lower alkyl, substituted or unsubstituted aryl or substituted or unsubstituted cycloalkyl) or —OR⁸ (wherein R⁸ represents hydrogen, substituted or unsubstituted lower alkyl, substituted or unsubstituted aryl or substituted or unsubstituted cycloalkyl); R² and R⁵ may be the same or different, and represent hydrogen, substituted or unsubstituted lower alkyl, a substituted or unsubstituted lower alkenyl, substituted or unsubstituted lower alkoxy, substituted or unsubstituted lower alkoxycarbonyl, substituted or unsubstituted aryl, carboxy, halogen, hydroxy, nitro, amino, or mono- or di-lower alkylamino; when n and m are 2 or 3, each of R² and R⁵ may be the same or different], a compound represented by the formula (II): 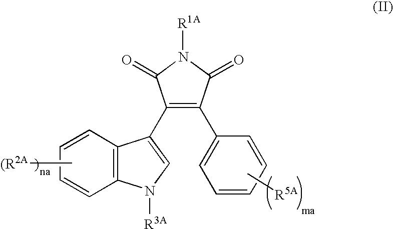
(wherein na, ma, R^{1A}, R^{2A}, R^{3A} and R^{5A} are as defined for the aforementioned n, m, R¹, R², R³ and R⁵, respectively) or a compound represented by the formula (III): 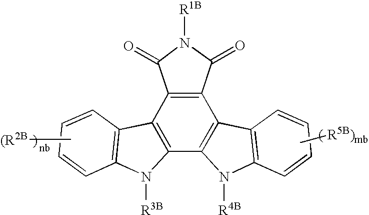
[wherein nb, mb, R^{1B}, R^{2B} and R^{5B} are as defined for the aforementioned n, m, R¹, R² and R⁵, respectively; R^{3B} and R^{4B} may be the same or different, and represent hydrogen, substituted or unsubstituted lower alkyl, substituted or unsubstituted lower alkenyl, —COR⁶ (wherein R⁶ is as defined above), —COOR⁷ (wherein R⁷ is as defined above) or —OR⁸(wherein R⁸ is as defined above), or R^{3B} and R^{4B} together form 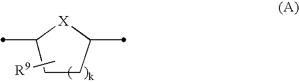
(wherein k represents 1 or 2; X represents CH₂, NH, an oxygen atom or a sulfur atom; R⁹ represents hydroxy, carboxy, carbamoyl or lower alkoxycarbonyl)], or a pharmacologically acceptable salt thereof.

(24) The agent for the promotion of neuropoiesis according to the above items (20) wherein the substance that inhibits the activity of GSK-3 is a compound represented by the formula (Ia): 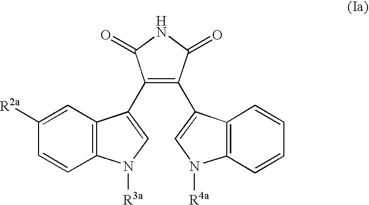
(wherein R^{2a} represents hydrogen, lower alkoxy, lower alkoxycarbonyl, aryl or nitro; R^{3a} and R^{4a} may be the same or different, and represent substituted or unsubstituted lower alkyl), or a pharmacologically acceptable salt thereof.

(25) The agent for the promotion of neuropoiesis according to the above item (20) wherein the substance that inhibits the activity of GSK-3 is a compound represented by the formula (IIa): 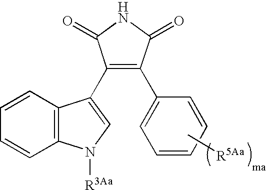
(wherein ma is as defined above; R^{3Aa} represents substituted or unsubstituted lower alkyl; R^{5Aa} represents halogen), or a pharmacologically acceptable salt thereof.

(26) The agent for the promotion of neuropoiesis according to the above item (20) wherein the substance that inhibits the activity of GSK-3 is a compound represented by the formula (IIIa): 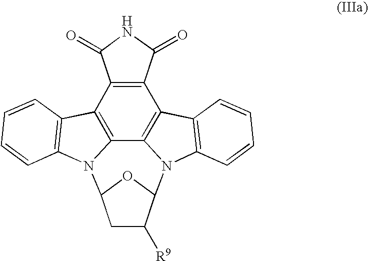
(wherein R⁹ is as defined above) or a pharmacologically acceptable salt thereof.

(27) The agent for the promotion of neuropoiesis according to the above item (20) wherein the substance that inhibits the activity of GSK-3 is a compound selected from the group consisting of 3,4-bis(1-methylindole-3-yl)-1H-pyrrole-2,5-dione, 3-(1-methylindole-3-yl)-4-(1-propylindole-3-yl)-1H-pyrrole-2,5-dione, 3-[1-(3-cyanopropyl)-indole-3-yl]-4-(1-methylindole-3-yl)-1H-pyrrole-2,5-dione, 3-[1-(3-aminopropyl)indole-3-yl]-4-(1-methylindole-3-yl)-1H-pyrrole-2,5-dione, 3-[1-(3-carboxypropyl)indole-3-yl]-4-(1-methylindole-3-yl)-1H-pyrrole-2,5-dione, 3-[1-(3-carbamoylpropyl)indole-3-yl]-4-(1-methylindole-3-yl)-1H-pyrrole-2,5-dione, 3-[1-(3-aminopropyl)indole-3-yl]-4-(1-methyl-5-propyloxyindole-3-yl)-1H-pyrrole-2,5-dione, 3-[1-(3-hydroxypropyl)indole-3-yl]-4-(1-methyl-5-phenylindole-3-yl)-1H-pyrrole-2,5-dione, 3-[1-(3-aminopropyl)indole-3-yl]-4-(1-methyl-5-phenylindole-3-yl)-1H-pyrrole-2,5-dione, 3-[1-(3-hydroxypropyl)indole-3-yl]-4-(1-methyl-5-methoxycarbonylindole-3-yl)-1H-pyrrole-2,5-dione, 3-[1-(3-hydroxypropyl)indole-3-yl]-4-(1-methyl-5-nitroindole-3-yl)-1H-pyrrole-2,5-dione, 3-(1-methylindole-3-yl)-4-[1-(3-hydroxypropyl)-5-nitroindole-3-yl]-1H-pyrrole-2,5-dione, 3-(2-chlorophenyl)-4-(1-methylindole-3-yl)-1H-pyrrole-2,5-dione, 3-(2,4-dichlorophenyl)-4-(1-methylindole-3-yl)-1H-pyrrole-2,5-dione, 3-(2-chlorophenyl)-4-[1-(3-hydroxypropyl)-indole-3-yl]-1H-pyrrole-2,5-dione, 4-[1-(3-aminopropyl)-indole-3-yl]-3-(2-chlorophenyl)-1H-pyrrole-2,5-dione and, 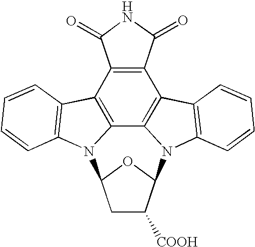
, or a pharmacologically acceptable salt thereof.

(28) The agent for the promotion of neuropoiesis according to the above item (20) wherein the substance that inhibits GSK-3 is a compound represented by the formula (IV): 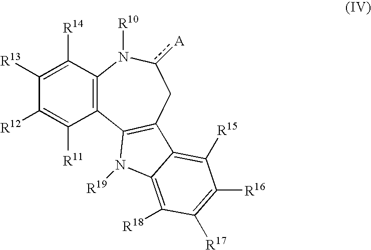
[wherein A is oxygen or sulfur coupled to the right by a single or double bond; R¹⁰ is selected from the group consisting of hydrogen, aryl, lower aliphatic substituents, particularly alkyl and lower alkyl ester; R¹¹-R¹⁴ are independently selected from the group consisting of alkoxy, amino, acyl, aliphatic substituents, particularly alkyl, alkenyl and alkinyl substituents, aliphatic alcohols, particularly alkyl alcohols, aliphatic nitriles, particularly alkyl nitriles, cyano, nitro, carboxyl, halogen, hydrogen, hydroxyl, imino and α,β-unsaturated ketones; R¹⁵-R¹⁸ are independently selected from the group consisting of aliphatic substituents, particularly alkyl, alkenyl and alkinyl substituents, particularly lower aliphatic substituents, alipahatic alcohols, particularly alkyl alcohols, alkoxy, acyl, cyano, nitro, epoxy, haloalkyl groups, halogen, hydrogen and hydroxyl; R¹⁹ is selected from the group consisting of aliphatic groups, particularly lower alkyl groups, aliphatic alcohols, particularly alkyl alcohols, carboxylic acids and hydrogen], or a pharmacologically acceptable salt thereof.

(29) The agent for the promotion of neuropoiesis according to the above item (20) wherein the substance that inhibits GSK-3 is a compound selected from the group consisting of 7,12-dihydro-indolo[3,2-d][1]benzazepin-6(5H)-one, 2-bromo-7,12-dihydro-indolo[3,2-d][1]benzazepin-6(5H)-one, 9-bromo-7,12-dihydro-indolo[3,2-d][1]benzazepin-6(5H)-one, 9-chloro-7,12-dihydro-indolo[3,2-d][1]benzazepin-6(5H)-one, 11-chloro-7,12-dihydro-indolo[3,2-d][1]benzazepin-6(5H)-one, 10-bromo-7,12-dihydro-indolo[3,2-d][1]benzazepin-6(5H)-one, 8-bromo-6,11-dihydro-thieno[3′,2′:2,3]azepino-[4,5-b]indol-5(4H)-one, 9-bromo-7,12-dihydro-4-methoxy-indolo[3,2-d][1]benzazepin-6(5H)-one, 9-bromo-7,12-dihydro-4-hydroxy-indolo[3,2-d][1]benzazepin-6(5H)-one, 7,12-dihydro-4-methoxy-indolo[3,2-d][1]benzazepin-6(5H)-one, 9-bromo-7,12-dihydro-2,3-dimethoxy-indolo[3,2-d]-[1]benzazepin-6(5H)-one, 9-bromo-7,12-dihydro-2,3-dihydroxy-indolo[3,2-d)[1]benzazepin-6(5H)-one, 7,12-dihydro-2,3-dimethoxy-indolo[3,2-d][1]benzazepin-6(5H)-one, 7,12-dihydro-9-trifluormethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2-bromo-7,12-dihydro-9-trifluoromethyl-indolo[3,2-d][1]benzazepin-6(5H)-one, 9-bromo-7,12-dihydro-indolo[3,2-d][1]benzazepin-6(5H)-thione, 9-bromo-5,12-bis-(t-butyloxycarbonyl)-7,12-dihydro-indolo[3,2-d][1]benzazepin-6(5H)-one, 9-bromo-12-(t-butyloxycarbonyl)-7,12-dihydro-indolo[3,2-d][1]benzazepin-6(5H)-one, 9-bromo-5,7-bis-(t-butyloxycarbonyl)-7,12-dihydro-indolo[3,2-d][1]benzazepin-6(5H)-one, 9-bromo-5,7,12-tri-(t-butyloxycarbonyl)-7,12-dihydro-indol o[3,2-d][1]benzazepin-6(5H)-one, 9-bromo-7,12-dihydro-5-methyloxycarbonylmethyl-indolo[3,2-d][1]benzazepin-6(5H)-one, 9-bromo-7,12-dihydro-12-methyloxycarbonylmethyl-indolo-[3,2-d][1]benzazepin-6(5H)-one, 9-bromo-7,12-dihydro-12-(2-hydroxyethyl)-indolo[3,2-d][1]benzazepin-6-(5H)-one, 2,9-dibromo-7,12-dihydro-indolo[3,2-d][1]-benzazepin-6(5H)-one, 8,10-dichloro-7,12-dihydro-indolo-[3,2-d][1]benzazepin-6(5H)-one, 9-cyano-7,12-dihydro-indolo[3,2-d][1]benzazepin-6(5H)-one, 9-bromo-7,12-di-hydro-5-methyl-indolo[3,2-d][1]benzazepin-6(5H)-one, 5-benzyl-9-bromo-7,12-dihydro-5-methyl-indolo[3,2-d][1]-benzazepin-6(5H)-one, 9-bromo-7,12-dihydro-12-methyl-indolo[3,2-d][1]benzazepin-6(5H)-one, 9-bromo-12-ethyl-7,12-dihydro-indolo[3,2-d][1]benzazepin-6(5H)-one, 9-bromo-7,12-dihydro-12-(2-propenyl)-indolo[3,2-d][1]benzazepin-6(5H)-one, 7,12-dihydro-9-methyl-indolo[3,2-d][1]-benzazepin-6(5H)-one, 7,12-dihydro-9-methoxy-indolo[3,2-d]-[1]benzazepin-6(5H)-one, 9-fluoro-7,12-dihydro-12-(2-propenyl)-indolo[3,2-d][1]benzazepin-6(5H)-one, 11-bromo-7,12-dihydro-indolo[3,2-d][1]benzazepin-6(5H)-one, 9-bromo-7,12-dihydro-2-(methyliminoamine)-indolo[3,2-d]-[1]benzazepin-6(5H)-one, 9-bromo-7,12-dihydro-2-(carboxylic acid)-indolo[3,2-d][1]benzazepin-6(5H)-one, 9-bromo-7,12-dihydro-10-hydroxy-indolo[3,2-d][1]benzazepin-6(5H)-one, 9-bromo-7,12-dihydro-11-hydroxymethyl-indolo[3,2-d][1]-benzazepin-6(5H)-one, 7,12-dihydro-4-hydroxy-indolo[3,2-d]-[1]benzazepin-6(5H)-one, 7,12-dihydro-2,3-dihydroxy-indolo[3,2-d][1]benzazepin-6(5H)-one, 2,3-dimethoxy-9-nitro-7,12-dihydro-indolo[3,2-d][1]benzazepin-6(5H)-one, 9-cyano-7,12-dihydro-indolo[3,2-d][1]benzazepin-6(5H)-one, 2,3-dimethoxy-9-cyano-7,12-dihydro-indolo[3,2-d][1]benzazepin-6(5H)-one, 9-nitro-7,12-dihydro-indolo[3,2-d][1]-benzazepin-6(5H)-one, 3-(6-oxo-9-trifluoromethyl-5,6,7,12-tetrahydro-indolo[3,2-d][1]benzazepin-2-yl)-propionitrile, 2-bromo-9-nitro-7,12-dihydro-indolo[3,2-d][1]benzazepin-6-(5H)-one, 3-(6-oxo-9-trifluoromethyl-5,6,7,12-tetrahydro-indolo[3,2-d][1]benzazepin-2-yl)acrylonitrile, 2-(3-hydroxy-1-propinyl)-9-trifluoromethyl-7,12-dihydro-indolo[3,2-d][1]benzazepin-6(5H)-one, 2-iodo-9-bromo-7,12-dihydro-indolo[3,2-d][1]benzazepin-6(5H)-one, 2-(3-oxo-1-butenyl)-9-trifluoromethyl-7,12-tetrahydro-indolo[3,2-d][1]benzazepin-6(5H)-one, 8-chloro-6,11-dihydro-thieno-(3′,2′:2,3]azepino[4,5-b]indol-5(4H)-one, 2-iodo-9-trifluoromethyl-7,12-dihydro-indolo[3,2-d][1]benzazepin-6-(5H)-one, 7,12-dihydro-pyrido[3′,2′:4,5]pyrrolo[3,2-d][1]-benzazepin-6(5H)-one, 11-methyl-7,12-dihydro-indolo-[3,2-d][1]-benzazepin-6(5H)-one, 2-[2-(1-hydroxycyclohexyl)-ethinyl]-9-trifluoromethyl-7,12-dihydro-indolo[3,2-d]-[1]benzazepin-6(5H)-one, 2-cyano-7,12-dihydro-indolo-[3,2-d][1]benzazepin-6(5H)-one, 2-iodo-7,12-dihydro-indolo[3,2-d][1]benzazepin-6(5H)-one, 11-ethyl-7,12-dihydro-indolo[3,2-d][1]benzazepin-6(5H)-one, 8-methyl-6,11-dihydro-thieno[3′,2′:2,3]azepino[4,5-b]indol-5(4H)-one and 3-(6-oxo-9-trifluoromethyl-5,6,7,12-tetrahydro-indolo[3,2-d][1]benzazepin-2-yl)acrylic acid, methyl ester, or a pharmacologically acceptable salt thereof.

(30) The agent for the promotion of neuropoiesis according to the above item (20) wherein the substance that inhibits GSK-3 is a compound selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]-benzazepin-6(5H)-one, 7,12-dihydro-9-trifluoromethyl-indolo[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indole-5(4H)-one, 7,12-dihydro-9-methoxy-indolo[3,2-d][1]benzazepin-6(5H)-one, 10-bromo-7,12-dihydro-indolo[3,2-d][1]benzazepin-6(5H)-one, 11-bromo-7,12-dihydro-indolo[3,2-d][1]benzazepin-6(5H)-one, 11-chloro-7,12-dihydro-indolo[3,2-d][1]-benzazepin-6(5H)-one, 9-fluoro-7,12-dihydro-indolo[3,2-d]-[1]benzazepin-6(5H)-one, 9-methyl-7,12-dihydro-indolo-[3,2-d][1]benzazepin-6(5H)-one, 9-bromo-7,12-dihydro-indolo[3,2-d][1]benzazepin-6(5H)-thione, 8,10-dichloro-7,12-dihydro-indolo[3,2-d][1]benzazepin-6(5H)-one, 9-bromo-7,12-dihydro-12-(2-hydroxyethyl)-indolo[3,2-d][1]benzazepin-6(5H)-one, 9-bromo-7,12-dihydro-2,3-dihydroxy-indolo[3,2-d][1]benzazepin-6(5H)-one, 2-bromo-7,12-dihydro-indolo[3,2-d][1]benzazepin-6(5H)-one, 7,12-dihydro-2,3-dimethoxy-indolo[3,2-d][1]benzazepin-6(5H)-one, 9-bromo-7,12-dihydro-12-methyl-indolo[3,2-d][1]benzazepin-6(5H)-one, 9-bromo-7,12-dihydro-5-methyloxycarbonylmethyl-indolo[3,2-d][1]benzazepin-6(5H)-one and 7,12-dihydro-indolo[3,2-d][1]benzazepin-6(5H)-one, or a pharmacologically acceptable salt thereof.

(31) The agent for the promotion of neuropoiesis according to the above item (20) wherein the substance that inhibits GSK-3 is a compound selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]-benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]-benzazepin-6(5H)-one, 7,12-dihydro-9-trifluormethyl-indolo[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indol-5(4H)-one and 7,12-dihydro-9-methoxy-indolo[3,2-d][1]benzazepin-6(5H)-one, or a pharmacologically acceptable salt thereof.

(32) The agent for the promotion of neuropoiesis according to the above item (20) wherein the substance that inhibits GSK-3 is 9-bromo-7,12-dihydro-indolo[3,2-d][1]-benzazepin-6(5H)-one or a pharmacologically acceptable salt thereof.

(33) The agent for the promotion of neuropoiesis according to the above item (20) wherein the substance that inhibits the activity of GSK-3 is a compound represented by the formula (V): 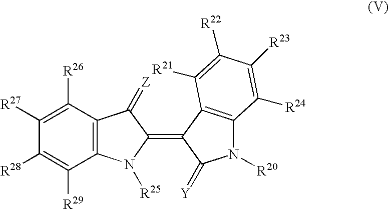
[wherein R²⁰ and R²⁵ which may be the same or different represent hydrogen; halogen; a hydroxy group; a methylene hydroxy group; a straight chain or branched C₁, to C₁₈-alkyl or alkoxy or methylenealkoxy group; a cycloalkyl group having 3 to 7 carbon atoms, and including one or more heteroatoms as needed; a substituted or unsubstituted aryl, aralkyl or aryloxy group having one or more heteroatoms as needed; a mono-, di- or trialkylsilyl group each independently having 1 to 6 carbon atoms within the straight chain or branched alkyl group; a mono-, di- or triarylsilyl group each independently having a substituted or unsubstituted aryl group; a trifluoromethyl group; —COM; —COOM; or a —CH₂COOM group (wherein M represents hydrogen, a straight chain or branched C₁ to C₁₈-alkyl group substituted with one or more hydroxy and/or amino groups if necessary, or an aryl group, which may be substituted with one or more halogen, alkyl groups or alkoxy groups, having one or more heteroatoms if necessary); an —NR³⁰R³¹ group (wherein R³⁰ and R³¹ which may be the same or different represent hydrogen, a C₁ to C₁₈ straight chain or branched alkyl group additionally substituted with one or more hydroxy and/or amino groups if necessary, a substituted or unsubstituted aryl group including one or more heteroatoms if necessary); an acyl group; a —CH₂—NR³⁰R³¹ methyleneamino group (wherein R³⁰ and R³¹ have the meanings as defined above); a benzyl group having one or more heteroatoms in the benzene ring if necessary; a methylenecycloalkyl group having 3 to 7 carbon atoms, and including one or more heteroatoms if necessary; a physiological amino acid group coupled to a nitrogen atom as an amide; an O-glycoside or N-glycoside of which glycoside being selected from monosaccharides or disaccharides; or a methylenesulfonate group; R²¹, R²², R²³, R²⁴, R²⁶, R²⁷, R²⁸ and R²⁹ which may be the same or different represent hydrogen; halogen; a hydroxy group; a nitroso group; a nitro group; an alkoxy group; a straight chain or branched C₁ to C₁₈ alkyl group substituted with one or more hydroxy and/or amino groups if necessary; a substituted or unsubstituted aryl group having one or more heteroatoms if necessary; a substituted or unsubstituted aralkyl group having one or more heteroatoms if necessary; a substituted or unsubstituted aryloxy group having one or more heteroatoms if necessary; a substituted or unsubstituted methylenearyloxy group having one or more heteroatoms if necessary; a cycloalkyl group having 3 to 7 carbon atoms, and including one or more heteroatoms if necessary; a methylenecycloalkyl group having 3 to 7 carbon atoms, and including one or more heteroatoms if necessary; a trifluoromethyl group; —COM; —COOM; or a CH₂COOM group (wherein M represents hydrogen, a straight chain or branched C₁ to C₁₈-alkyl group additionally substituted with one or more hydroxy and/or amino groups if necessary, or an aryl group, which may be substituted with one or more halogen atoms, alkyl groups or alkoxy groups, having one or more heteroatoms if necessary); an —NR³⁰R³¹ group (wherein R³⁰ and R³¹ which may be the same or different represent hydrogen, a straight chain or branched C₁ to C₁₈-alkyl group additionally substituted with one or more hydroxy and/or amino groups if necessary, a substituted or unsubstituted aryl group including one or more heteroatoms if necessary, an acyl group; or form apart of cycloalkyl having 3 to 7 carbon atoms with the nitrogen atom including one or more heteroatoms if necessary); a —CONR³⁰R³¹ group (wherein R³⁰ and R³¹ have the meanings as defined above); a hydroxylamino group; a phosphate group; a phosphonate group; a sulfate group; a sulfonate group; a sulfonamide group; an —SO₂NR³⁰R³¹ group (wherein R³⁰ and R³¹ have the meanings as defined above); an —N═N—R³² azo group (wherein R³² represents an aromatic group substituted with one or more carboxyl, phosphoryl or sulfonate groups if necessary, or an O-glycoside or N-glycoside group of which glycoside being selected from monosaccharides or disaccharides); or R²⁰ and R²⁴, and R²⁵ and R²⁹ together form a ring having one to four CH₂ groups each independently substituted if necessary, respectively; Y and Z which may be the same or different represent an oxygen; sulfur; selenium; tellurium atom; an NR³³ group (wherein R³³ represents hydrogen, a straight chain or branched C₁ to C₁₈ alkyl group substituted with one or more carboxyl, phosphoryl or sulfonate groups if necessary, a substituted or unsubstituted aryl group including one or more heteroatoms if necessary, an aralkyl group or a sulfonate group); or —NOR³³ (wherein R³³ group have the meanings as defined above)], or a pharmacologically acceptable salt thereof.

(34) The agent for the promotion of neuropoiesis according to the above item (20) wherein the substance that inhibits GSK-3 is a compound selected from the group consisting of indirubin, 5-iodo-indirubin, 5-bromo-indirubin, 5-chloro-indirubin, 5-fluoro-indirubin, 5-methyl-indirubin, 5-nitro-indirubin, 5-SO₃H-indirubin, 5′-bromo-indirubin, 5-5′-dibromo-indirubin and 5′-bromo-indirubin 5-sulfonic acid, or a pharmacologically acceptable salt thereof.

(35) The agent for the promotion of neuropoiesis according to the above item (20) wherein the substance that inhibits GSK-3 is a compound selected from the group consisting of indirubin-3′-monooxime, 5-iodo-indirubin-3′-monooxime and 5-SO₃Na-indirubin-3′-monooxime, or a pharmacologically acceptable salt thereof.

(36) The agent for the promotion of neuropoiesis according to the above item (20) wherein the substance that inhibits GSK-3 is indirubin-3′-monooxime or a pharmacologically acceptable salt thereof.

(37) A neuron obtained by culturing a neural stem cell in the presence of the agent for the promotion of neuropoiesis according to any one of the above items (20) to (36).

(38) A method of the manufacture of a neuron which comprises culturing a neural stem cell in the presence of the agent for the promotion of neuropoiesis according to any one of the above items (20) to (36) to allow neogenesis of the neuron, and collecting the neuron from the culture.

(39) A method of the regeneration of a nerve which comprises administering a substance that inhibits GSK-3.

(40) Use of a substance that inhibits GSK-3 for the manufacture of a nerve regenerating drug.

(41) Use of a substance that inhibits GSK-3 for the manufacture of an agent for the promotion of neuropoiesis of a neural stem cell.

Hereinafter, details of the present invention are explained.

1. A Substance that Inhibits the Activity of GSK-3 Included in the Nerve Regenerating Drug and the Agent for the Promotion of Neuropoiesis of a Neural Stem Cell of the Invention

The substance that inhibits the activity of GSK-3 may be any one as long as it is a compound that inhibits the activity of GSK-3, and examples thereof include e.g., lithium, bisindolylmaleimide derivatives, 3-aryl-4-indolylmaleimide derivatives, indolocarbazole derivatives, indolo[3,2-d][1]benzazepin-6(5H)-one derivatives, indirubin derivatives and the like.

Specific examples of the bisindolylmaleimide derivative, 3-aryl-4-indolylmaleimide derivative, indolocarbazole derivative include e.g., compounds represented by the formulae (I) to (III). Among them, 3,4-bis(1-methylindole-3-yl)-1H-pyrrole-2,5-dione, 3-(1-methylindole-3-yl)-4-(1-propylindole-3-yl)-1H-pyrrole-2,5-dione, 3-[1-(3-cyanopropyl)-indole-3-yl]-4-(1-methylindole-3-yl)-1H-pyrrole-2,5-dione, 3-[1-(3-aminopropyl)indole-3-yl]-4-(1-methylindole-3-yl)-1H-pyrrole-2,5-dione, 3-[1-(3-carboxypropyl)indole-3-yl]-4-(1-methylindole-3-yl)-1H-pyrrole-2,5-dione, 3-[1-(3-carbamoylpropyl)indole-3-yl]-4-(1-methylindole-3-yl)-1H-pyrrole-2,5-dione, 3-[1-(3-aminopropyl)indole-3-yl]-4-(1-methyl-5-propyloxyindole-3-yl)-1H-pyrrole-2,5-dione, 3-[1-(3-hydroxypropyl)indole-3-yl]-4-(1-methyl-5-phenylindole-3-yl)-1H-pyrrole-2,5-dione, 3-[1-(3-aminopropyl)indole-3-yl]-4-(1-methyl-5-phenylindole-3-yl)-1H-pyrrole-2,5-dione, 3-[1-(3-hydroxypropyl)indole-3-yl]-4-(1-methyl-5-methoxycarbonylindole-3-yl)-1H-pyrrole-2,5-dione, 3-[1-(3-hydroxypropyl)indole-3-yl]-4-(1-methyl-5-nitroindole-3-yl)-1H-pyrrole-2,5-dione, 3-(1-methylindole-3-yl)-4-[1-(3-hydroxypropyl)-5-nitroindole-3-yl]-1H-pyrrole-2,5-dione, 3-(2-chlorophenyl)-4-(1-methylindole-3-yl)-1H-pyrrole-2,5-dione, 3-(2,4-dichlorophenyl)-4-(1-methylindole-3-yl)-1H-pyrrole-2,5-dione, 3-(2-chlorophenyl)-4-[1-(3-hydroxypropyl)indole-3-yl]-1H-pyrrole-2,5-dione, 3-[1-(3-aminopropyl)indole-3-yl]-4-(2-chlorophenyl)-1H-pyrrole-2,5-dione, 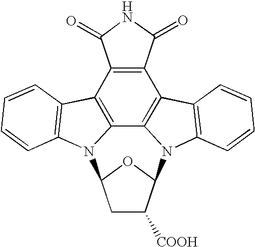
and the like are preferred.

Specific examples of the indolo[3,2-d][1]benzazepin-6(5H)-one derivative include e.g., compounds represented by the formula (IV). Among them, 7,12-dihydro-indolo[3,2-d][1]benzazepin-6(5H)-one, 2-bromo-7,12-dihydro-indolo[3,2-d][1]benzazepin-6(5H)-one, 9-bromo-7,12-dihydro-indolo[3,2-d][1]benzazepin-6(5H)-one, 9-chloro-7,12-dihydro-indolo[3,2-d][1]benzazepin-6(5H)-one, 11-chloro-7,12-dihydro-indolo[3,2-d][1]benzazepin-6(5H)-one, 10-bromo-7,12-dihydro-indolo[3,2-d][1]benzazepin-6(5H)-one, 8-bromo-6,11-dihydro-thieno[3′,2′:2,3]azepino[4,5-b]-indole-5(4H)-one, 9-bromo-7,12-dihydro-4-methoxy-indolo-[3,2-d][1]benzazepin-6(5H)-one, 9-bromo-7,12-dihydro-4-hydroxy-indolo[3,2-d][1]benzazepin-6(5H)-one, 7,12-dihydro-4-methoxy-indolo[3,2-d][1]benzazepin-6(5H)-one, 9-bromo-7,12-dihydro-2,3-dimethoxy-indolo[3,2-d][1]-benzazepin-6(5H)-one, 9-bromo-7,12-dihydro-2,3-dihydroxy-indolo-[3,2-d][1]benzazepin-6(5H)-one, 7,12-dihydro-2,3-dimethoxy-indolo[3,2-d][1]benzazepin-6(5H)-one, 7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2-bromo-7,12-dihydro-9-trifluoromethyl-indolo[3,2-d][1]benzazepin-6(5H)-one, 9-bromo-7,12-dihydro-indolo[3,2-d][1]-benzazepin-6(5H)-thione, 9-bromo-5,12-bis-(t-butyloxycarbonyl)-7,12-dihydro-indolo[3,2-d][1]benzazepin-6(5H)-one, 9-bromo-12-(t-butyloxycarbonyl)-7,12-dihydro-indolo-[3,2-d][1]benzazepin-6(5H)-one, 9-bromo-5,7-bis-(t-butyloxycarbonyl)-7,12-dihydro-indolo[3,2-d][1]benzazepin-6-(5H)-one, 9-bromo-5,7,12-tri-(t-butyloxycarbonyl)-7,12-dihydro-indol o[3,2-d][1]benzazepin-6(5H)-one, 9-bromo-7,12-dihydro-5-methyloxycarbonylmethyl-indolo[3,2-d][1]benzazepin-6(5H)-one, 9-bromo-7,12-dihydro-12-methyloxycarbonylmethyl-indolo[3,2-d][1]benzazepin-6(5H)-one, 9-bromo-7,12-dihydro-12-(2-hydroxyethyl)-indolo[3,2-d][1]-benzazepin-6(5H)-one, 2,9-dibromo-7,12-dihydro-indolo[3,2-d]-[1]benzazepin-6(5H)-one, 8,10-dichloro-7,12-dihydro-indolo-[3,2-d][1]benzazepin-6(5H)-one, 9-cyano-7,12-dihydro-indolo[3,2-d][1]benzazepin-6(5H)-one, 9-bromo-7,12-dihydro-5-methyl-indolo[3,2-d][1]benzazepin-6(5H)-one, 5-benzyl-9-bromo-7,12-dihydro-5-methyl-indolo[3,2-d][1]-benzazepin-6(5H)-one, 9-bromo-7,12-dihydro-12-methyl-indolo[3,2-d][1]benzazepin-6(5H)-one, 9-bromo-12-ethyl-7,12-dihydro-indolo[3,2-d][1]benzazepin-6(5H)-one, 9-bromo-7,12-dihydro-12-(2-propenyl)-indolo[3,2-d][1]benzazepin-6(5H)-one, 7,12-dihydro-9-methyl-indolo[3,2-d][1]-benzazepin-6(5H)-one, 7,12-dihydro-9-methoxy-indolo[3,2-d][1]benzazepin-6(5H)-one, 9-fluoro-7,12-dihydro-12-(2-propenyl)-indolo[3,2-d][1]benzazepin-6(5H)-one, 11-bromo-7,12-dihydro-indolo[3,2-d][1]benzazepin-6(5H)-one, 9-bromo-7,12-dihydro-2-(methyliminoamine)-indolo[3,2-d][1]benzazepin-6(5H)-one, 9-bromo-7,12-dihydro-2-(carboxylic acid)-indolo[3,2-d][1]benzazepin-6(5H)-one, 9-bromo-7,12-dihydro-10-hydroxy-indolo[3,2-d][1]benzazepin-6(5H)-one, 9-bromo-7,12-dihydro-11-hydroxymethyl-indolo[3,2-d][1]-benzazepin-6(5H)-one, 7,12-dihydro-4-hydroxy-indolo[3,2-d][1]benzazepin-6(5H)-one, 7,12-dihydro-2,3-dihydroxy-indolo-[3,2-d][1]benzazepin-6(5H)-one, 2,3-dimethoxy-9-nitro-7,12-dihydro-indolo[3,2-d][1]benzazepin-6(5H)-one, 9-cyano-7,12-dihydro-indolo[3,2-d][1]benzazepin-6(5H)-one, 2,3-dimethoxy-9-cyano-7,12-dihydro-indolo[3,2-d][1]benzazepin-6(5H)-one, 9-nitro-7,12-dihydro-indolo[3,2-d][1]-benzazepin-6(5H)-one, 3-(6-oxo-9-trifluoromethyl-5,6,7,12-tetrahydro-indolo[3,2-d][1]benzazepin-2-yl)propionitrile, 2-bromo-9-nitro-7,12-dihydro-indolo[3,2-d][1]benzazepin-6(5H)-one, 3-(6-oxo-9-trifluoromethyl-5,6,7,12-tetrahydro-indolo[3,2-d][1]benzazepin-2-yl)acrylonitrile, 2-(3-hydroxy-1-propinyl)-9-trifluoromethyl-7,12-dihydro-indolo[3,2-d][1]benzazepin-6(5H)-one, 2-iodo-9-bromo-7,12-dihydro-indolo[3,2-d][1]benzazepin-6(5H)-one, 2-(3-oxo-1-butenyl)-9-trifluoromethyl-7,12-tetrahydro-indolo[3,2-d][1]benzazepin-6(5H)-one, 8-chloro-6,11-dihydro-thieno[3,2′:2,3]-azepino[4,5-b]indole-5(4H)-one, 2-iodo-9-trifluoromethyl-7,12-dihydro-indolo[3,2-d.][1]benzazepin-6(5H)-one, 7,12-dihydro-pyrido[3′,2′:4,5]pyrrolo[3,2-d][1]-benzazepin-6(5H)-one, 11-methyl-7,12-dihydro-indolo-[3,2-d][1]benzazepin-6(5H)-one, 2-[2-(1-hydroxycyclohexyl)ethinyl]-9-trifluoromethyl-7,12-dihydro-indolo-[3,2-d][1]benzazepin-6(5H)-one, 2-cyano-7,12-dihydro-indolo[3,2-d][1]benzazepin-6(5H)-one, 2-iodo-7,12-dihydro-indolo[3,2-d][1]benzazepin-6(5H)-one, 11-ethyl-7,12-dihydro-indolo[3,2-d][1]benzazepin-6(5H)-one, 8-methyl-6,11-dihydro-thieno[3′,2′:2,3]azepino[4,5-b]indole-5(4H)-one, 3-(6-oxo-9-trifluoromethyl-5,6,7,12-tetrahydro-indolo-[3,2-d][1]benzazepin-2-yl)acrylic acid, methyl ester and the like are preferred.

Specific examples of the indirubin derivative include e.g., compounds represented by the formula (V). Among them, indirubin, 5-iodo-indirubin, 5-bromo-indirubin, 5-chloro-indirubin, 5-fluoro-indirubin, 5-methyl-indirubin, 5-nitro-indirubin, 5-SO₃H-indirubin, 5′-bromo-indirubin, 5-5′-dibromo-indirubin, 5′-bromo-indirubin 5-sulfonic acid, indirubin-3′-monooxime, 5-iodo-indirubin-3′-monooxime, 5-SO₃Na-indirubin-3′-monooxime and the like are preferred.

Hereinafter, compounds represented by the formulae (I) to (V) are referred to as compounds (I) to (V), respectively. The same is applicable to compounds represented by other number of the formula.

In the definition of each group in the compounds (I) to (III) and the compounds (Ia) to (IIIa), followings are illustrated.

(i) Examples of lower alkyl moiety of the lower alkyl, lower alkoxy and lower alkoxycarbonyl include e.g., straight chain or branched alkyl having 1 to 10 carbon atoms, and specific examples thereof include methyl, ethyl, propyl, isopropyl, butyl, isobutyl, sec-butyl, tert-butyl, pentyl, isopentyl, neopentyl, hexyl, heptyl, octyl, 6-methylheptyl, isooctyl, nonyl, decyl and the like.

(ii) Examples of the cycloalkyl include e.g., cycloalkyl having 3 to 8 carbon atoms, and specific examples thereof include cyclopropyl, cyclobutyl, cyclopentyl, cyclohexyl, cycloheptyl, cyclooctyl and the like.

(iii) Examples of the lower, alkenyl include e.g., straight chain, branched or cyclic alkenyl having 2 to 8 carbon atoms, and specific examples thereof include vinyl, allyl, 1-propenyl, butenyl, pentenyl, hexenyl, heptenyl, octenyl, cyclohexenyl, 2,6-octadienyl and the like.

(iv) Examples of lower alkyl moiety of the mono- or di-lower alkylamino are as defined for the aforementioned lower alkyl, and two lower alkyl moieties of a dilower alkylamino may be the same or different.

(v) The halogen represents each atom of fluorine, chlorine, bromine and iodine.

(vi) Examples of the aryl include e.g., monocyclic, bicyclic or tricyclic aryl having 6 to 14 carbon atoms, and specific examples thereof include phenyl, naphthyl, indenyl, anthranil and the like.

(vii) The substituents in the substituted lower alkyl, substituted lower alkenyl, substituted lower alkoxy and substituted lower alkoxycarbonyl may be the same or different, and examples thereof include halogen, cyano, nitro, hydroxy, carboxy, carbamoyl, amino, mono- or di-lower alkylamino, cycloalkyl, lower alkanoyl, lower alkoxy, aryl, substituted aryl, aryloxy, substituted aryloxy, lower alkoxycarbonyl, lower alkanoyloxy and the like with the number of substitution being 1 to 3.

The illustrated halogen, mono- or di-lower alkylamino, cycloalkyl, aryl moiety of the aryl and aryloxy, and the lower alkyl moiety of the lower alkoxy, lower alkoxycarbonyl, lower alkanoyl and lower alkanoyloxy herein are as defined for the aforementioned halogen (v), mono- or di-lower alkylamino (iv), cycloalkyl (ii), aryl (vi) and lower alkyl (i).

Further, the illustrated substituents in the substituted aryl and substituted aryloxy herein may be the same or different, and examples thereof include lower alkyl, lower alkoxy, lower alkoxycarbonyl, halogen, cyano, nitro, hydroxy, carboxy, amino and the like with the number of substitution being 1 to 3.

The illustrated halogen, and the lower alkyl moiety of the lower alkyl, lower alkoxy and lower alkoxycarbonyl herein are as defined for the aforementioned halogen (v) and lower alkyl (i).

(viii) Examples of the substituent in the substituted aryl and substituted cycloalkyl include e.g., lower alkyl, substituted lower alkyl and the like in addition to the groups included in the definition of the substituent in the aforementioned substituted lower alkyl (vii).

The illustrated substituents in the substituted lower alkyl herein may be the same or different, and examples thereof include e.g., lower alkoxy, lower alkoxycarbonyl, halogen, cyano, nitro, hydroxy, carboxy, amino and the like with the number of substitution being 1 to 3.

The illustrated halogen and the lower alkyl moiety of the lower alkoxy and lower alkoxycarbonyl herein are as defined for the aforementioned halogen (v) and lower alkyl (i).

Further, the illustrated lower alkyl herein is as defined for the aforementioned lower alkyl (i).

Pharmacologically acceptable salts of the bisindolylmaleimide derivative, 3-aryl-4-indolylmaleimide derivative, indolocarbazole derivative, indolo[3,2-d][1]-benzazepin-6(5H)-one derivative, indirubin derivative, compounds (I) to (V) and compounds (Ia) to (IIIa) are preferably nontoxic and water soluble, and examples thereof include e.g., inorganic acid salts such as hydrochlorides, sulfates, nitrates and phosphates, and organic acid salts such as acetates, maleates, fumarates and citrates; examples of pharmacologically acceptable metal salt include alkaline metal salts such as sodium salts and potassium salts, alkaline earth metal salts such as magnesium salts and calcium salts, aluminum salts, zinc salts and the like; examples of pharmacologically acceptable ammonium salt include salts such as ammonium and tetramethylammonium; examples of pharmacologically acceptable organic amine salt include addition salts of morpholine, piperidine and the like; and the like.

The bisindolylmaleimide derivative, 3-aryl-4-indolylmaleimide derivative, indolocarbazole derivative, indolo[3,2-d][1]benzazepin-6(5H)-one derivative, indirubin derivative, compounds (I) to (V) and compounds (Ia) to (IIIa) can be produced by the method described in EP 470490, WO 93/18766, WO 93/18765, EP 397060, WO 98/11105, WO 98/11103, WO 98/11102, WO 98/04552, WO 98/04551, DE 4243321, DE 4005970, DE 4217964, DE 4005970, DE 3914764, WO 96/04906, WO 95/07910, DE 42179464, U.S. Pat. No. 5,856,517, U.S. Pat. No. 5,891,901, WO 99/42100, EP 328026, EP 384349, EP 540956, DE 4005969, EP 508792, WO 99/65910, WO 01/037819 and the like, or the method according to the same.

Moreover, as the substance that inhibits the activity of GSK-3, a short interference RNA (siRNA) can be also used. The siRNA for GSK-3 suppresses the expression of GSK-3 by the RNAi activity thereof, and consequently, it can inhibit the activity of GSK-3. The siRNA can inhibit the activity of GSK-3 through introducing itself within a cell, and in addition, the inhibition also becomes possible by introducing a vector that expresses the siRNA into a cell.

For producing an effective siRNA on human GSK-3, it is important to select a target sequence that is highly effective. A variety of methods have been known as an algorithm for the determination of the sequence, however, for example, a highly effective target sequence can be selected among 19 arbitrary sequences in a messenger RNA sequence of human GSK-3, by selecting a sequence that fulfills more of the respective conditions of: content of guanine and cytosine being 30 to 52%; three or more bases being adenine or uridine among 5 bases at 3′ end; melting temperature being less than 20° C.; the third base being adenine; the tenth base being uridine; the 13th base being other than glycine; the 19th base being adenine; the 19th base not being glycine and cytosine. An effective siRNA for human GSK-3 can be produced by allowing hybridization of both of a sense stranded oligo RNA prepared by adding nucleotides of two bases at 3′ end of an oligo RNA having the target sequence, and an antisense stranded oligo RNA prepared by adding nucleotides of two bases at 3′ end of an oligo RNA having a sequence that is complementary to the target sequence. Although synthesis, purification and hybridization of the siRNA can be executed by any one of various methods, for example, they can be performed using Silencer siRNA Construction Kit (manufactured by Ambion, Inc.) according to the attached protocol. The vector for expressing the siRNA can be any one of various kinds of plasmid vectors, and various kinds of viral vectors such as retroviral vectors, lentiviral vectors and adenoviral vectors. For example, it can be produced by incorporating the oligo DNA having the target sequence selected by the aforementioned method into piGENE hU6 Vector (manufactured by iGENE Therapeutics, Inc.) according to the attached protocol. Although introduction of the siRNA or the vector that expresses an siRNA into a cell can be executed by any one of a variety of methods, for example, it can be performed using Nucleofector Device (manufactured by Amaxa) according to the attached protocol.

2. Method of Searching a substance that Inhibits the Activity of GSK-3

Exemplary method of searching a substance that inhibits the activity of GSK-3 includes a method which comprises:

[i] permitting coexistence of GSK-3, a peptide phosphorylated by GSK-3, and ATP, in the presence of a subject substance,

[ii] permitting coexistence of GSK-3, a peptide phosphorylated by GSK-3, and ATP as in the above [i] in the absence of a subject substance,

[iii] measuring and comparing the amount of thus phosphorylated peptides in both cases [i] and [ii], and

[iv] selecting a substance yielding a smaller amount of the phosphorylated peptide in the presence of the subject substance, in comparison with the absence of the subject substance.

Although the subject substance is not particularly limited, examples thereof include e.g., peptides; proteins; cell extracts and purified products derived from the cell extract; cell culture supernatants and purified products derived from the supernatant; biological samples such as a serum and purified products derived from the biological sample; somatic extracts of a microorganism and purified products derived from the somatic extract; microorganism culture supernatants and purified products derived from the supernatant; compounds; compounds synthesized using combinatorial chemistry; and the like.

GSK-3 is not particularly limited as long as it has the activity of GSK-3, but examples thereof include GSK-3α or β derived from preferably a mammal, and more preferably rat, mouse, monkey or human, and specific examples include a protein having an amino acid sequence set out in SEQ ID NO: 1.

GSK-3 can be obtained by a method which comprises introducing an expression vector having a gene encoding GSK-3 into an animal cell, and culturing the animal cell, and the like. The gene encoding GSK-3 is not particularly limited as long as it encodes GSK-3, but examples thereof include genes encoding GSK-3α or β derived from preferably a mammal, and more preferably rat, mouse, monkey or human, and specific examples include a gene having a base sequence set out in SEQ ID NO: 2.

Examples of the peptide phosphorylated by GSK-3 include glycogen synthase, and examples of the glycogen synthase include e.g., a peptide having an amino acid sequence set out in SEQ ID NO: 3.

The glycogen synthase can be obtained by a method which comprises introducing an expression vector having a gene encoding glycogen synthase into an animal cell, and culturing the animal cell, and the like. The gene encoding glycogen synthase is not particularly limited as long as it is a gene encoding glycogen synthase, but examples thereof include genes encoding glycogen synthase derived from preferably a mammal, and more preferably rat, mouse, monkey or human, and specific examples include a gene having a base sequence set out in SEQ ID NO: 4.

Furthermore, a peptide having an amino acid sequence including a site which is phosphorylated by GSK-3 can be also used as the peptide phosphorylated by GSK-3 in the amino acid sequence of an eukaryotic initiation factor 2B (eIF2B) protein which participates in translation of a protein, and specifically, examples thereof include a peptide having an amino acid sequence set out in SEQ ID NO: 5.

Exemplary method of the measurement of the activity of GSK-3 may include e.g., a method which comprises subjecting glycogen synthase or a peptide including a phosphorylation site of the enzyme to a phosphorylation reaction by GSK-3 using [γ-³³P]ATP as ATP that may be a donor of phosphoric acid in the presence of a subject substance, or in the absence of the subject substance; and measuring the amount of ³³P incorporated into the enzyme or peptide using a liquid scintillation counter or the like.

3. Nerve Regenerating Drug

A nerve regenerating drug refers to an agent exhibiting an action to increase an intracerebral neuron by promoting neuropoiesis through directly acting on an intracerebral neural stem cell of a human or an animal.

The nerve regenerating drug can be used as a therapeutic drug for neurological diseases accompanied by degeneration or damage of a nerve.

Examples of the neurological disease include Parkinson's disease, Alzheimer's disease, Down's disease, cerebrovascular disorder, cerebral stroke, spinal cord injury, Huntington's chorea, multiple sclerosis, amyotrophic lateral sclerosis, epilepsy, anxiety disorder, schizophrenia, depression, manic depressive psychosis and the like.

The substance that inhibits the activity of GSK-3, or a pharmacologically acceptable salt thereof can be administered neat alone as a nerve regenerating drug, however, it is desired that to provide as any one of various medical drug formulations, in general. Furthermore, those medical drug formulations are used for animals and humans.

The nerve regenerating drug of the invention may contain, as an active ingredient, a substance that inhibits the activity of GSK-3 or a pharmacologically acceptable salt thereof alone, or in a mixture with other arbitrary active ingredient for therapy. Further, those medical drug formulations are manufactured by an arbitrary method which has been well known in the technical field of physical pharmacy through mixing the active ingredient with one or more pharmacologically acceptable carriers.

Route of administration for use is desirably a route which is the most effective for the therapy, and examples thereof include oral or parenteral such as e.g., intravenous route.

Examples of dosage form include tablet, powder, granule, syrup, injection and the like.

Liquid preparations such as e.g., syrups suited for oral administration can be produced using water, a saccharide such as sucrose, sorbit or fructose, a glycol such as polyethylene glycol or propylene glycol an oil such as sesame oil, olive oil or soybean oil, a preservative such as p-hydroxybenzoate ester, a flavor such as strawberry flavor or peppermint. Furthermore, the tablet, powder granule and the like can be produced using an excipient such as lactose, glucose, sucrose or mannit, a disintegrant such as starch or sodium alginate, a lubricant such as magnesium stearate or talc, a binder such as polyvinyl alcohol, hydroxypropyl cellulose or gelatin, a surfactant such as fatty acid ester, a plasticizer such as glycerin or the like.

Formulation which is suitable for the parenteral administration preferably comprises a sterile aqueous agent containing an active compound that is isotonic with the blood of a recipient. For example, in cases of an injection, a solution for injection is prepared using a carrier comprising a saline solution, a glucose solution or a mixture of a saline solution and a glucose solution, and the like.

Moreover, also in cases of these parenteral formulations, one or more auxiliary components as illustrated for the oral formulation selected from diluents, preservatives, flavors, excipients, disintegrants, lubricants, binders, surfactants, plasticizers and the like can be added.

Amount of administration and frequency of administration of the substance that inhibits the activity of GSK-3 or a pharmacologically acceptable salt thereof may vary depending on the dosage form, age, body weight of the patient, nature or severity of the symptom to be treated, however, in cases of oral administration, 0.01 mg to 1 g, preferably 0.05 to 50 mg per one adult is generally administered once or several times per day. In cases of parenteral administration such as intravenous administration, 0.001 to 100 mg, preferably 0.01 to 10 mg per one adult is generally administered once or several times per day.

4. Agent for the Promotion of Neuropoiesis of Neural Stem Cell

An agent for the promotion of neuropoiesis of a neural stem cell refers to an agent that promotes neuropoiesis of a neural stem cell when it is brought into contact with a neural stem cell in vivo or in vitro.

The neural stem cell is not particularly limited as long as it is a neural stem cell, but cerebral adult neural stem cells are preferred.

The brain may be a brain of any animals, however, examples thereof include brains of preferably mammals, and more preferably, rat, mouse, monkey, human and the like.

Although the substance that inhibits the activity of GSK-3 or a pharmacologically acceptable salt thereof can be used neat alone as an agent for the promotion of neuropoiesis of a neural stem cell, it is usually desired that the substance is provided as any kind of medical formulations. Further, those medical formulations are used for animals as well as humans.

The agent for the promotion of neuropoiesis of a neural stem cell of the invention may contain the substance that inhibits the activity of GSK-3 or a pharmacologically acceptable salt thereof alone, as an active ingredient, or as a mixture with other arbitrary active ingredient for the therapy. Those medical formulations can be manufactured by a similar method to that for the formulation of the aforementioned nerve regeneration drug, and can be administered according to any method of administration which is similar thereto.

The agent for the promotion of neuropoiesis of a neural stem cell of the invention can be used for a method of the manufacture of a neuron which comprises bringing the agent into contact with a neural stem cell in vitro, culturing the nerve cell to permit neogenesis of a neuron and collecting the neuron from the culture. When the agent for the promotion of neuropoiesis of a neural stem cell of the invention is used in vitro, it is preferred that the substance that inhibits the activity of GSK-3 or a pharmacologically acceptable salt thereof is used after dissolving in a solution capable of dissolving the substance or the salt. Examples of the solution include water, DMSO and the like.

5. Neuron Obtained by Culturing a Neural Stem Cell in the Presence of the Agent for the Promotion of Neuropoiesis of the Invention

Through culturing an animal neural stem cell in the presence of the agent for the promotion of neuropoiesis of the invention, neuropoiesis of the neural stem cell can be actively promoted. The animal neural stem cell may be a neural stem cell of any animal, and examples thereof include neural stem cells derived from preferably mammals, more preferably rat, mouse, monkey and human. The neural stem cell may be a neural stem cell derived from a brain. Although the neural stem cell may be a cell derived from an animal with any week old or year old, however, preferably, it may be an adult neural stem cell.

Examples of the process for obtaining an adult neural stem cell from an animal include a process which comprises excising a brain from an adult animal by a surgical procedure, preparing a brain cell crude extract, and concentrating adult stem cells from the crude extract, according to the process described in J. Neuroscinece, 19, 8487 (1999) and Genes & Develop., 10, 3129 (1996).

Further, examples of the process for obtaining an adult neural stem cell from a human include a process which comprises collecting a tissue from lateral ventricle wall of a patient suffering from a neurological disease by biopsy, preparing a brain cell crude extract, and concentrating adult stem cells from the extract, according to the method described in Experimental Cell Research, 289, 378 (2003).

When the adult neural stem cell is cultured in the presence of the agent for the promotion of neuropoiesis of the invention, it is preferred that the agent for the promotion of neuropoiesis is brought into action at a concentration of 100 nmol/l to 100 mmol/l per the adult neural stem cell at a density of approximately 1.8×10⁵ cells/cm². However, the lithium or a pharmacologically acceptable salt thereof is preferably brought into action at a concentration of 100 μmol/l to 10 mmol/l. Neuropoiesis can be promoted through bringing the adult neural stem cell into contact with the agent for the promotion of neuropoiesis of the invention, and allowing static culture at 37° C. in an atmosphere of 5% CO₂ for 4 to 14 days while conducting medium replacement of total amount or partial amount every three days.

The medium for use in the culture of the adult neural stem cell may be any medium as long as it does not prevent the promotion of neuropoiesis, however, it is preferred that a DMEM/F12 medium (manufactured by Invitrogen. Corporation) containing a 1% N2 supplement (manufactured by Invitrogen-Corporation) or the like is used.

The neuron obtained by the aforementioned culture can be used for the therapy of the neurological disease by recovering from the culture medium and transplanting to an impaired part of a patient suffering from the neurological disease by a surgical procedure. Examples of the neurological disease include Parkinson's disease, Alzheimer's disease, Down's disease, cerebrovascular disorder, cerebral stroke, spinal cord injury, Huntington's chorea, multiple sclerosis, amyotrophic lateral sclerosis, epilepsy, anxiety disorder, schizophrenia, depression, manic depressive psychosis and the like.

6. Method of Evaluation of the Nerve Regenerating Drug of the Invention

Possible therapy of a neurological disease by the nerve regenerating drug of the invention through regeneration of a neuron in vivo can be verified by the following method.

The aforementioned nerve regenerating drug of the invention is administered to an experimental animal such as rat or monkey. Although the experimental animal may be a healthy animal without injury, an animal with a brain injured by a method such as ischaemia, administration of 6-hydroxydopamine (6-OHDA) or administration of kainic acid is preferred because neuropoiesis can be effectively observed by inflicting an ischemic injury of hippocampus [Cell, 110, 429 (2002)]. The route of administration may be oral, intraoral, subcutaneous, intramuscular, intravenous or intraventricular route. In connection with amount of administration and method of administration, for example, administration in an amount of 100 μg to 10 mg, preferably 500 μg to 500 ng per 1 kg of the body weight, preferably once or several times per day is conducted. In the case of parenteral administration such as intravenous administration, administration in an amount of 10 μg to 1 mg, preferably 100 μg to 100 ng per 1 kg of the body weight, preferably once or several times per day is conducted.

The neuron yielded by neogenesis can be detected by the following method.

After administering a retroviral vector capable of expressing a gene having an ability to label a cell such as Green Fluorescent Protein (GFP) or beta galactosidase, or bromodeoxyuridine (BrdU) which can label proliferating cells to the experimental animal concurrently with, prior to or following the initial administration of the substance, the substance is administered once or several times per day, and the animal is kept for 10 to 20 days. Thereafter, brain of the experimental animal is excised, and the cerebral frozen section is prepared. The section is observed using a fluorescence microscope, and for example, in the case where BrdU is used as the agent that labels proliferating cells, number of BrdU positive cells and ratio of the number of BrdU positive cells to the number of Tuj1 positive cells which is a neuron marker are compared to those for a negative control per unit area.

According to the method as described above, the nerve regenerating drug of the invention can be evaluated for the neuropoiesis promoting action and therapeutic effect for a neurological disease.

7. Method of Evaluation of the Agent for the Promotion of Neuropoiesis of the Invention

ANSC-7 cells which can be obtained by the method described in Reference Example 2 are plated at 1.8×10⁵ cells per well in a 12-well culture dish which was coated with polyornithine and laminin and was filled with a DMEM/F12 medium containing 1 ml of a 1% N2 supplement (manufactured by Invitrogen Corporation) and 20 ng/ml FGF-2 (manufactured by PeproTech Inc.), and incubated overnight. Entire culture fluid is changed into a DMEM/F12 medium containing 0.5% fetal bovine serum and a 1% N2 supplement without including FGF2 (manufactured by Invitrogen Corporation, hereinafter, referred to as differentiation inducing medium) to induce differentiation. Upon the induction, the substance that inhibits the activity of GSK-3 serially diluted in PBS (manufactured by Invitrogen Corporation) or DMSO in the range of from 0.01 mmol/l to 5 mol/l is added thereto in 1/1000 volume. As a negative control, the same volume of PBS or DMSO is added.

After changing the culture fluid into the differentiation inducing medium supplemented with each substance that inhibits the activity of GSK-3 every three days, and inducing differentiation for 6 days, it is replaced with a 15% neutral buffered formalin solution (Wako Pure Chemical Industries, Ltd.) to allow fixation for 20 min. Thereafter, washing with PBS containing 0.3% Triton X-100 (manufactured by Nacalai Tesque, Inc.) for 5 min is repeated three times. Next, after blocking the cells using a 10% goat fetal serum diluted in PBS (manufactured by DAKO Corporation) for 2 hrs, a reaction is allowed with a mouse anti-Tuj1 (β tubulin isotype III) antibody (manufactured by Sigma-Aldrich Corporation) diluted 1000 times in PBS as a primary antibody at 4° C. for 16 hrs. Thereafter, washing with PBS containing 0.3% Triton X-100 for 5 min is repeated three times.

Next, a reaction is allowed with Alexa Fluor 488 conjugate goat anti-mouse IgG antibody (manufactured by Molecular Probes, Inc.) diluted 1000 times as a secondary antibody at room temperature for 2 hrs. At the same time, Bisbenzimide H 33342 Fluorochrome, Trihydrochloride (manufactured by CALBIOCHEM, hereinafter, described as H33342) is added thereto to give the final concentration of 1 μg/ml to stain the nucleus. After immersing in PBS, observation with an inverted fluorescence microscope (manufactured by Nikon Corporation) is conducted, and the number of Tuj1 positive cells per 2.44 mm² is counted.

Hereinafter, Experimental Example relating to a neuropoiesis promoting action of the agent for the promotion of neuropoiesis of the invention is demonstrated.

### EXPERIMENTAL EXAMPLE 1

### Promotion of Neuropoiesis with Lithium Chloride (1)

According to the method described in the above section 7, when the differentiation of the ANSC-7 cells is induced, lithium chloride or sodium chloride (both manufactured by Nacalai Tesque, Inc.) dissolved in PBS to give 0.01, 0.1, 1 or 3 mol/l is added to the medium containing the ANSC-7 cells in 1/1000 volume of the culture fluid, and the number of neurons on day 6 following the induction of differentiation was analyzed. As a consequence, the number of Tuj1 positive neurons became 1.1, 1.3, 1.8 and 2.1 times with lithium chloride at the final concentration of 0.01, 0.1, 1 and 3 mmol/l, respectively (significantly different with lithium chloride of 1 mmol/l or greater), exhibiting increase depending on the concentration of lithium chloride. Furthermore, total number of H33342 positive cells with 3 mmol/l lithium chloride was 1.1 times compared to the control without lithium chloride, exhibiting no significant difference. As in the foregoing, it was revealed that lithium chloride exhibits a neuropoiesis promoting action of an ANSC-7 cell. Moreover, in the case of sodium chloride that is a negative control, the number of Tuj1 positive neurons is 1.0, 1.1, 1.2, 1.2 times at the final concentration of 0.01, 0.1, 1, 3 mmol/l, respectively, all of which not being significantly different. Thus, increase in the number of neogenetic neurons is believed not being the effect due to salt concentration or chloride ion, but being the effect of lithium.

### EXPERIMENTAL EXAMPLE 2

### Promotion of Neuropoiesis by Lithium Chloride (2)

In order to elucidate if the neuropoiesis promoting action on the ANSC-7 cell results from the induction of differentiation of BDNF and Bcl-2, whether the expression of BDNF and Bcl-2 is promoted by lithium or not was analyzed with semiquantitative RT-PCR.

The ANSC-7 cells were plated in 7 wells in total of a 6-well culture dish which had been coated with polyornithine and laminin and had been filled with a DMEM/F12 medium containing 2 ml of a 1% N2 supplement and 20 ng/ml FGF-2 to give 4.5×10⁵ cells per well, and incubated overnight. Total RNA was obtained from cells in one well using RNeasy mini kit (manufactured by QIAGEN), according to the accompanying protocol. Entire culture medium in the remaining 6 wells was changed into the differentiation inducing medium to induce the differentiation. To two wells among those was added 3 mol/l of lithium chloride in 1/1000 volume of the medium, and to other two wells was added 1 mol/l of lithium chloride in 1/1000 volume of the medium. Equal volume of PBS wad added to the remaining two wells as a control.

After 24 hrs passed from initiation of the induction of differentiation, cells were harvested from each well to which lithium chloride at each concentration was added. Total RNA was obtained from the cells according to a similar method to that described above, and from the remaining cells was obtained total RNA on day 6 following initiation of the induction of differentiation. To the total RNA each in an amount of 5 μg obtained as described above were added 10 μl of 5×DNAse buffer, 0.5 μl of an RNase inhibitor (40 U/μl), 2.5 μl of RNase-free DNaseI (1 U/μl) (all of which manufactured by Promega Corporation), respectively, and sterile water was added thereto to give the total volume of 50 μl. After allowing a reaction at 37° C. for 30 min, the mixture was subjected to a phenol/chloroform treatment followed by ethanol precipitation.

To the total RNA each in an amount of 1 μg which had been subjected to the DNase treatment was added 1 μl of 0.5 μg/μl oligo(dT)12-18 primer, and sterile water was added thereto to give the total volume of 12 μl. After heating at 65° C. for 10 min, the mixture was rapidly cooled on ice, and thereto were added 4 μl of 5× synthesis buffer (manufactured by Invitrogen Corporation), 1 μl of 10 mmol/l dNTP mix, 2 μl of 0.1 mol/l DTT, 1 μl of 200 U/μl Superscript II RT (manufactured by Invitrogen Corporation) to allow a reaction at 42° C. for 50 min. After heating the mixture at 90° C. for 5 min, it was left to stand on ice for 10 min.

Next, 1 μl of RNaseH (2 U/μl) (manufactured by Invitrogen Corporation) was added thereto, and a reaction was allowed at 37° C. for 20 min. Accordingly, a cDNA was produced by adding sterile water thereto to give the total volume of 200 μl.

In a similar manner, a cDNA was prepared from rat cerebral total RNA for use as a positive control. To 1 μl of the cDNA were added 2 μl of a 10 μmol/l primer set, 1 μl of DMSO (manufactured by Nacalai Tesque, Inc.), 2 μl of 10×ExTaq buffer, 1.6 μl of dNTPmix, 0.1 μl of ExTaq (all of which manufactured by TAKARA BIO INC.), and each cDNA fragment was amplified after treating at 94° C. for 1 min, through repeating 27 cycles of PCR for Bcl-2 amplification and 35 cycles of PCR for BDNF amplification of: at 94° C. for 1 min, at 60° C. for 1 min, and at 74° C. for 1 min using a thermal cycler. For the amplification of Bcl-2, synthetic DNAs consisting of the base sequence set out in SEQ ID NOs: 6 and 7 were used; and for amplification of BDNF, synthetic DNAs consisting of the base sequence set out in SEQ ID NOs: 8 and 9 were used as a primer set.

The amplified DNA was electrophoresed on a 1.8% agarose (manufactured by Nacalai Tesque, Inc.) gel, and after staining with ethidium bromide (Manufactured by Nacalai Tesque, Inc.), detection was executed with a transilluminator (manufactured by Toyobo Co., Ltd.). Strength of the Bcl-2 band did not vary depending on the difference in concentration of lithiumchloride both 24 hrs and 6 days following initiation of the differentiation. Expression of BDNF was not found regardless of the difference in concentration of lithium chloride.

From the results described above, it was suggested that the neuropoiesis promoting activity by lithium chloride was not the result of suppression of cell death via Bcl-2 and BDNF, but lithium chloride actively promoted neuropoiesis.

### EXPERIMENTAL EXAMPLE 3

### Promotion of Neuropoiesis by Lithium Chloride (3)

In order to elucidate if the neuropoiesis promoting action by lithium chloride results from the increase in newly generated cells due to suppression of apoptosis, or from the active induction of differentiation of a neuron, the effect of apoptosis suppression against ANSC-7 cells by lithium was analyzed.

According to the method described in the above section 7, lithium chloride was added to give the final concentration of 3 mmol/l into the medium containing ANSC-7 cells followed by culture for 6 days. ANSC-7 cells thus cultured after adding lithium chloride, and ANSC-7 cells cultured after adding PBS as a control were allowed to a reaction using an in situ cell death detection kit, fluorescein (manufactured by Roche Diagnostics K.K.) according to the attached protocol. The cells were observed using an inverted fluorescence microscope (manufactured by Nikon Corporation), and the number of apoptotic cell was counted per 2.44 mm².

Consequently, the number of apoptosis positive cells was 1.0 time through adding lithium chloride, which results in no alteration. Therefore, it was elucidated that lithiumchloride did not suppress the apoptosis of ANSC-7 cells. Accordingly, the neuropoiesis promoting action by lithium was revealed to result from active promotion of neuropoiesis.

### EXPERIMENTAL EXAMPLE 4

### Antagonistic Action for Neuropoiesis Promoting Action of Insulin and Forskolin, and Lithium

Antagonistic action for neuropoiesis promoting action of insulin and forskolin known as having a neuropoiesis promoting action, and lithium was analyzed.

According to the method described in the above section 7, insulin was added to give the concentration in the medium of 5 μg/ml or 25 μg/ml into the medium upon the induction of differentiation of the ANSC-7 cells, accompanied by adding lithium chloride to the medium containing each concentration of insulin to give the final concentration of 0, 1 and 3 mmol/l. Then, induction of the differentiation was allowed for 6 days.

Consequently, rate of increase in neurons caused by 3 mmol/l of lithium chloride was 0.70 time lower in the case of coexistence with 25 μg/ml of insulin compared to the case of coexistence with 5 μg/ml of insulin, exhibiting a significant decrease. Therefore, antagonism of insulin and lithium was revealed.

Furthermore, according to a similar method to that described above, forskolin was added to the medium in stead of insulin to give the final concentration of 0, 1 and 5 μmol/l, and the rate of increase in neurons in the case of coexistence with lithium chloride at a final concentration of 0 or 3 mmol/l was calculated.

Consequently, rate of increase in neurons caused by 1, 5 μmol/l of forskolin under the condition without lithium chloride was 1.9, 2.2 times, respectively, whereas in the case of coexistence with 3 mmol/l of lithium chloride, it was 1.2, 1.1 times, leading to no increase. Therefore, lithiumchloride and forskolin were revealed to antagonize for the neuropoiesis promoting action.

As a target molecule of lithium, GSK-3, inositol-1-phosphate phosphatase and inositol-polyphosphatase have been known [Nature, 417, 292-295 (2002)]. Furthermore, insulin and forskolin have been known to indirectly inhibit the activity of GSK-3 [Mol. Cell. Biol., 19, 4989-5000 (1999)]. Because the target molecule of lithium, insulin and forskolin for the neuropoiesis promoting action is assumes to be common, it was suggested that lithium promoted neuropoiesis of a neural step cell by inhibiting the activity of GSK-3.

### EXPERIMENTAL EXAMPLE 5

### Promotion of Neuropoiesis by SB-216763 that is a Selective Inhibitor of GSK-3

SB-216763 known as a selective inhibitor of GSK-3 synthesized according to the method described in Reference Example 1 [Chem. Biol., 7, 793-803 (2000)] was dissolved in DMSO to give 0.1 and 0.33 mmol/l, which was added to a medium containing ANSC-7 in 3/1000 volume thereof upon induction of the differentiation of ANSC-7 cells, according to the method described in the above section 7. Thus, the number of neurons on day 6 following induction of the differentiation was measured.

Consequently, the number of Tuj1 positive neurons became 1.2 times and 1.8 times by SB-216763 at the final concentration of 0.3 and 1.0 μmol/l, respectively, exhibiting significant increase in a concentration dependent manner. Therefore, it was revealed that neuropoiesis can be promoted by a compound having the activity to selectively inhibit GSK-3.

As in the foregoing, the substance having the activity to selectively inhibit GSK-3 was suggested to be an agent for the promotion of neuropoiesis of a neural stem cell, as well as a medicament for nerve regenerative therapy.

### EXPERIMENTAL EXAMPLE 6

### Promotion of Neuropoiesis by 9-bromo-7,12-dihydro-indolo[3,2-d][1]benzazepin-6(5H)-one (Kenpaullone) that is an Inhibitor of GSK-3

According to a similar method to that shown in Experimental Example 1, Kenpaullone which has been known as an inhibitor of GSK-3 and cyclin-dependent kinase (hereinafter, referred to as CDK) [Biochem. J., 371, 199-204 (2003), manufactured by CALBIOCHEM] was dissolved in DMSO to give 0.5, 2 and 5 mmol/l. The DMSO solution in the 1/1000 volume of the culture fluid was added to the medium containing the ANSC-7 cells. Thus, the number of Tuj1 positive neurons on day 6 following induction of the differentiation was analyzed. As a negative control, an equal volume of DMSO was added. Moreover, in a similar manner, Roscovitine which has been known as a CDK inhibitor and which hardly inhibits GSK-3 [Biochem. J., 371, 199-204 (2003), manufactured by Sigma-Aldrich Corporation] was dissolved to give 2,5 and 10 mmol/l in DMSO. The DMSO solution in the 1/1000 volume of the culture fluid was added to the medium containing ANSC-7 cells, and thus, the number of Tuj1 positive neurons on day 6 following induction of the differentiation was analyzed.

Consequently, the number of Tuj1 positive neurons was 1.3 times with Kenpaullone at the final concentration of 0.5 μmol/l, 2.7 times at 2 μmol/l and 3.7 times at 5 μmol/l, in comparison with the negative control, exhibiting significant increase in a concentration dependent manner of Kenpaullone. On the other hand, it was 1.0 time with Roscovitine at the final concentration of 2 μmol/l, 1.2 times at 5 μmol/l, and 1.1 times at 10 μmol/l, in comparison with the negative control, exhibiting no significant increase, respectively. Thus, no increase in the number of Tuj1 neurons caused by Roscovitine was found. Therefore, it was revealed that Kenpaullone exhibited a neuropoiesis promoting action, and that the action is not caused by an inhibitory activity on CDK of Kenpaullone, but is caused by an inhibitory activity on GSK-3.

As in the foregoing, it was suggested that any compound having the activity to inhibit GSK-3 may be an agent for the promotion of neuropoiesis of a neural stem cell, as well as a medicament for nerve regenerative therapy of a neurological disease, not being limited to lithium and SB-216763.

### EXPERIMENTAL EXAMPLE 7

### Suppression of Neuropoiesis by High Expression of a GSK-3β Gene

Because high expression of GSK-3β is found in the brain of a patient suffering from Alzheimer's disease [J. Neuropathol. Exp. Neurol., 56, 70-78 (1997)], influences of the high expression of GSK-3β on differentiation of a neuron of an adult neural stem cell were studied using a retroviral vector in order to elucidate the relationship between the caue of onset of Alzheimer's disease and GSK-3β.

First, a cDNA encoding a wild type rat GSK-3β gene was prepared as follows using a cDNA derived from a rat brain described in Experimental Example 2 as a template. To 2.5 μl of the template cDNA were added 3 μl of a 10 μmol/l primer set including the sequences set out in SEQ. ID NOs: 10 and 11 (manufactured by Proligo LLC), 5 μl of 10×PCR buffer for KOD-plus-, 5 μl of 2 mmol/l dNTPs, 2 μl of 25 mmol/l MgSO₄, 1 μl of KOD-plus-DNA polymerase (all of which manufactured by Toyobo Co., Ltd.) and 31.5 μl of sterile water, and then amplification of the cDNA fragment was allowed by incubating at 94° C. for 2 min followed by repeating 25 cycles of: at 94° C. for 15 sec, at 60° C. for 30 sec, and at 68° C. for 1 minute and 20 sec using a thermal cyler.

On the other hand, a cDNA encoding a mutated rat GSK-3β gene having a mutation of a lysine residue at position 85 into an arginine residue, which mutation may lead to loss of the kinas activity of GSK-3β [Proc. Nat. Acad. Sci. USA, 92,8498-8502 (1995)] was similarly prepared as follows.

With 2.5 μl of a cDNA encoding a wild type rat GSK-3β gene as a template, amplification of a cDNA having a partial length of 5′ end of the mutated rat GSK-3β was allowed using a primer set including the sequence set out in SEQ ID NOs: 10 and 12, while amplification of a cDNA having a partial length of 3′ end of the mutated rat GSK-3β was allowed using a primer set including the sequence set out in SEQ ID NOs: 11 and 13. With a mixture of respective cDNAs having the partial length as a template, a cDNA having the full length of the mutated rat GSK-3β was amplified using a primer set including the sequence set out in SEQ ID NOs: 10 and 11.

Each of the amplification reaction fluid of wild type and mutant was subjected to a treatment with phenol/chloroform followed by precipitation through adding ethanol. The product was purified with a QIAquick PCR purification kit (manufactured by QIAGEN) according to the attached protocol.

Subsequently, to 10 μg of a pCLNCX plasmid vector (manufactured by IMGENEX Corporation) were added 10 μl of 10×M buffer and 5 μl of HindIII (manufactured by TAKARABIO INC.), and thereto was added sterile water to give 100 μl. The reaction was allowed at 37° C. for 12 hrs. After subjecting the reaction fluid to a treatment with phenol/chloroform, the DNA was precipitated by adding ethanol and was dissolved in 32 μl of sterile water. To the DNA solution were added 4 μl of 10× Blunting buffer and 4 μl of KOD DNA polymerase to allow a reaction at 72° C. for 2 min to achieve blunting. After treating the reaction fluid with phenol/chloroform, thereto was added ethanol to precipitate the DNA. After dissolving the DNA in 43 μl of sterile water, thereto were added 5 μl of 10× Alkaline Phosphatase buffer and 2 μl of Alkaline Phosphatase (all of which manufactured by TAKARA BIO INC.), and the reaction was allowed at 65° C. for 30 min. After treating the reaction fluid with phenol/chloroform, thereto was added ethanol to precipitate the DNA which was dissolved in sterile water.

With 3 μg of thus cleaved pCLNCX plasmid vector was mixed 3 μg of the cDNA encoding the wild type or mutant rat GSK-3β gene prepared as described above, to which sterile water was added to give 4 μl. Thereto was added 4 μl of ligation high (manufactured by Toyobo Co., Ltd.) and the reaction was allowed at 16° C. for 12 hrs. The product was transformed into E. coli DH5α competent cell (manufactured by TAKARA BIO INC.). An ampicillin resistant colony was cultured in a liquid LB medium according to a conventional method, and a pCLNC-GSK3β plasmid DNA and a pCLNC-GSK3β (K85R) plasmid DNA were prepared using a DNA Endofree Plasmid Maxi Kit (manufactured by QIAGEN) according to the attached protocol.

Next, a viral vector was produced as follows, and a function relating to the differentiation of a neuron of ANSC-7 cells was analyzed. First, 15 μg of pCLNC-GSK3β, pCLNC-GSK3β (K85R), or pCLNCX plasmid vector DNA as a negative control, and 5 μg of each pMD.G plasmid vector DNA (supplied from Salk Institute, USA) were dissolved in 2 ml of a D-MEM high glucose medium (manufactured by Invitrogen Corporation), and transfection was carried out using a Transfast transfection reagent (manufactured by Promega Corporation) according to the attached protocol into 293 gp cells (supplied from Salk Institute, USA) which had been prepared on the preceding day.

On day 3 following the transfection, the culture supernatant was filtrated through a 0.45 μm filter (manufactured by Millipore Corporation) to recover a solution containing the viral vector. The viral vector solution was transferred into a polyaroma tube (manufactured by Hitachi Koki Co., Ltd.), and centrifuged using an ultracentrifuge (manufactured by Hitachi Koki Co., Ltd.) at 50,000×g, 18° C. for 90 min. The supernatant was eliminated, and the precipitated viruses were suspended in a DMEM/F12 medium containing a 1% N2 supplement and 20 ng/ml FGF-2 and 8 μg/ml hexadimethrin bromide (manufactured by Sigma-Aldrich Corporation). According to a similar method to that shown in Experimental Example 1, 1.8×10⁵ ANSC-7 cells per well were plated on a 12-well culture dish which had been coated with polyornithine and laminin, and left to stand overnight. Culture supernatant was removed from the culture, and thereto was added the viral suspension to allow infection by the culture in an incubator at 37° C., at the concentration of CO₂ of 5% for 2 hrs. Subsequently, the entire culture fluid was changed to a differentiation inducing medium to initiate the induction of differentiation. Similarly to Experimental Example 1, the number of Tuj1 positive neurons on day 6 following induction of the differentiation was analyzed.

Consequently, compared to ANSC-7 cells infected with the retrovirus which had been produced from pCLNCX as a negative control and allowed differentiation, the number of neogenetic neurons was significantly decreased by 33% in the cells infected with a retrovirus which had been produced from pCLNC-GSK3β to permit high expression of GSK3β and allowed differentiation. On the other hand, in the cells infected with a retrovirus which had been produced from pCLNC-GSK3β (K85R) to permit high expression of GSK3β (K85R) having no kinase activity and allowed differentiation, the number of neogenetic neurons was significantly greater by 34% than in the cells in which high expression of wild type GSK-3β was allowed, which was almost the same level as the case in which the retrovirus which had been produced from pCLNCX as a negative control was infected and allowed differentiation. Therefore, it was revealed that neuropoiesis was suppressed by the kinase activity of GSK-3β.

Accordingly, a possibility of onset of Alzheimer's disease through suppression of neuropoiesis due to promotion of phosphorylation of a target molecule which results from high expression of GSK-3β, thereby causing suppression of cerebral autoregeneration ability, was suggested. Thus, it was indicated that GSK-3 inhibitors can be a therapeutic drug for neurological diseases such as e.g., Alzheimer's disease.

### EXPERIMENTAL EXAMPLE 8

### Suppression of Neuropoiesis by an Associated Beta Amyloid Peptide, and Relief of Suppression by GSK-3 Inhibitor (1)

A beta amyloid peptide (hereinafter, referred to as Aβ) is a principal component constituting senile plaques, and it is a substance which has been believed to be a cause of Alzheimer's disease [Proc. Nat. Acad. Sci., USA, 98, 11039-11041 (2001)]. Aβ [1-40] (manufactured by BIOSOURCE) was dissolved in 0.1% (v/v) trifluoroacetic acid (Manufactured by Nacalai Tesque, Inc.) to give 10 mg/ml, and incubated at 25° C. for 1 hour. Thereafter, the solution was diluted in PBS to give 0.5 mg/ml. The diluted solution was incubated at 25° C. for 24 hrs to allow the formation of an associated product [J. Biol. Chem., 276, 42027-42034 (2001)]. According to a similar method to that shown in Experimental Example 1, the associated product was added to the medium to give the final concentration of 0.1 mg/ml upon induction of the differentiation of the ANSC-7 cell, while an equal volume of PBS was added for the negative control. On day 2 and day 4 following the induction of differentiation, the entire medium was changed to the differentiation inducing medium alone, respectively, and the number of neurons was measured on day 6 following induction of the differentiation.

Consequently, the number of Tuj1 positive neurons significantly decreased by 78% through the addition of the associated Aβ. Therefore, a possibility of onset of Alzheimer's disease that is one of neurological diseases, through suppression of neuropoiesis due to the associated Aβ, thereby causing suppression of cerebral autoregeneration ability, was suggested.

### EXPERIMENTAL EXAMPLE 9

### Suppression of Neuropoiesis by an Associated Beta Amyloid Peptide, and Relief of Suppression by GSK-3 Inhibitor (2)

In order to elucidate if suppression of neuropoiesis by associated Aβ is caused by decrease in the number of neogenetic cells resulting from promotion of apoptosis, or if it is caused by suppression of neuronal differentiation, the effect of promoting apoptosis on an ANSC-7 cell exerted by associated Aβ was analyzed.

According to a similar method to that shown in Experimental Example 8, ANSC-7 cells subjected to induction of differentiation for 6 days by a differentiation inducing medium containing the associated Aβ at the final concentration of 0.1 mg/ml, and ANSC-7 cells subjected to induction of differentiation by a medium containing PBS as a control were allowed to react using an in situ cell death detection kit, fluorescein (manufactured by Roche Diagnostics K.K.) according to the attached protocol. The cells were observed using an inverted fluorescence microscope (manufactured by Nikon Corporation), and the number of apoptotic cells per 2.44 mm² was counted.

Consequently, the number of apoptotic positive cells increased through the addition of the associated Aβ by 14%, but any significant difference was found. Therefore, it was revealed that decrease in the number of neogenetic neurons due to the associated Aβ was primarily caused by suppression of neuronal differentiation rather than promotion of apoptosis.

### EXPERIMENTAL EXAMPLE 10

### Suppression of Neuropoiesis by an Associated Beta Amyloid Peptide, and Relief of Suppression by GSK-3 Inhibitor (3)

According to a similar method to that shown in Experimental Example 8, lithiumchloride at a final concentration of 3 mmol/l, or Kenpaullone at a final concentration of 2 μmol/l was added to a differentiation inducing medium containing the associated Aβ at a final concentration of 0.1 mg/ml upon inducing differentiation of the ANSC-7 cell, and the number of Tuj1 positive neurons on day 6 following inducing differentiation was analyzed.

As a result, by adding lithium chloride or Kenpaullone, the number of Tuj1 positive neurons significantly increased by 73%, 400%, respectively, in comparison with the case of the associated Aβ alone.

Therefore, it was revealed that a GSK-3 inhibitor exhibited an action to release, suppression of neuropoiesis due to the associated Aβ.

As in the foregoing, it was indicated that compounds having the activity to selectively inhibit GSK-3 could be a nerve regenerating drug for neurological diseases such as Alzheimer's disease, and that the compounds could be agent for the promotion of neuropoiesis of a neural stem cell.

### EXPERIMENTAL EXAMPLE 11

### Promotion of Neuropoiesis by Indirubin-3′-monoxime, an Inhibitor of GSK-3

According to a similar method to that shown in Experimental Example 1, indirubin-3′-monoxime that has been known as an inhibitor of GSK-3 [J. Biol. Chem., 276, 251-260 (2001), manufactured by Sigma-Aldrich Corporation] was dissolved in DMSO to give 1 mmol/l, and the DMSO solution in the 1/1000 volume of the culture fluid was added to a medium containing ANSC-7 cells. Thus, the number of Tuj1 positive neurons on day 6 following induction of the differentiation was analyzed. As a negative control, an equal volume of DMSO was added. Consequently, under the condition of the addition of indirubin-3′-monoxime at a final concentration of 1 μmol/l, the number of Tuj1 positive neurons significantly increased 1.4 times in comparison with the negative control. Therefore, it was revealed that indirubin-3′-monoxime exhibited a neuropoiesis promoting action.

From the foregoings, compounds having the activity to inhibit GSK-3, not being limited to lithium, SB-216763 and Kenpaullone, can be an agent for the promotion of neuropoiesis of a neural stem cell, as well as a medicament for nerve regenerative therapy of a neurological disease.

### EXPERIMENTAL EXAMPLE 12

### Promotion of Neuropoiesis by Specific siRNA for GSK-3

A short interference RNA (siRNA) specifically knocks down a gene [Nature, 411, 494-498 (2001)]. In order to further elucidate possible the capability to promote the neuropoiesis through specifically inhibiting GSK-3 which is expressed in an adult neural stem cell, a chemically synthesized siRNA that is specific for GSK-3β was introduced into an ANSC-7 cell, and studied for effects on neuropoiesis.

First, a knockdown effect of two kinds of chemically synthesized siRNA on GSK-3β in an ANSC-7 cell was studied as follows. Introduction of the siRNA into an ANSC-7 cell was carried out using Rat NSC Nucleofector™ Kit (manufactured by Amaxa). Double stranded siRNA-B1 having SEQ ID NO: 14 and 15, or double stranded siRNA-B2 having SEQ ID NO: 16 and 17 (manufactured by Dharmacon, Inc.) in an amount of 150 pmol per 1×10⁶ of ANSC-7 cells was introduced according to the attached protocol. As a negative control, Non-specific Control Duplex IX (47% GC Content) siRNA (manufactured by Dharmacon, Inc.) was introduced. Immediately after the introduction, they were suspended in 5 ml of a differentiation inducing medium, and seeded on a 6 cm culture dish coated with polyornithine and laminin. At 48 hrs following the seeding, the cells were recovered by dissolving in an aqueous solution containing 1% Nonidet P-40, 50 mM Tris-HCl (pH7.4), 50 mM NaCl (all of which manufactured by Nacalai Tesque, Inc.) and one tablet of Complete mini, EDTA free (manufactured by Roche Diagnostics K.K.) per 10 ml. The amount of GSK-3β was detected by a Western blotting method after separating by an SDS-PAGE process according to a conventional method. For the detection, an anti-GSK-3 antibody that recognized GSK-3α and β (manufactured by Sigma Corporation) was used. As a result, the cells to which two kinds of siRNAs for GSK-3β were introduced both exhibited a decreased amount of GSK-3β by 90% or greater in comparison with the negative control, but the amount of GSK-3α was unchanged. Therefore, it was revealed that each siRNA could specifically, knock down GSK-3β.

Subsequently, after introducing those two kinds of siRNAs into ANSC-7, the cells were immediately seeded at 1.8×10⁵ cells on a 12-well culture dish which had been coated with polyornithine and laminin and filled with 1 ml of a differentiation inducing medium to induce the differentiation. According to a similar method to that shown in Experimental Example 1, the number of Tuj1 positive neurons on day 6 following induction of the differentiation was analyzed. As a result, the number of Tuj1 positive neurons increased 2.5 times in cells to which siRNA-B1 was introduced in comparison with the negative control, while in cells to which siRNA-B2 was introduced, the number increased 1.9 times. Therefore, it was revealed that the siRNA for GSK-3β siRNA exhibited a neuropoiesis promoting action.

From the foregoing, it was suggested that compounds having the activity to inhibit GSK-3, and nucleic acids including the siRNA could be an agent for the promotion of neuropoiesis of a neural stem cell, as well as a medicament for nerve regenerative therapy of neurological diseases.

### REFERENCE EXAMPLE 1

### Step 1 of Synthesis of 3-(2,4-dichlorophenyl)-4-(1-methylindole-3-yl)-1H-pyrrole-2,5-dione (SB-216763): Synthesis of 3-indole glyoxylic acid, methyl ester

Commercially available 3-indole glyoxylic acid (9.55 g) was suspended in methylene chloride (300 mL), and cooled on ice. Thereafter, to the suspension was added oxalyl chloride (8.8 mL), followed by stirring at 20° C. for 20 hrs. The reaction fluid was cooled on ice, and after adding methanol (190 mL) thereto, the reaction fluid was stirred at 25° C. for 1 hour. To the reaction fluid were added water and methylene chloride, and thus deposited crystals were filtrated, followed by washing of the crystals with methylene chloride. The crystals were dried under a reduced pressure to obtain 3-indole glyoxylic acid, methyl ester (7.07 g, 69%).

### Step 2: Synthesis of 2-(1-methylindole-3-yl)-2-oxoacetic acid, methyl ester

The 3-indole glyoxylic acid, methyl ester (5.88 g) obtained in the step 1 was dissolved in N,N-dimethylformamide (180 mL), and thereto was added hydrogenated sodium (dispersed in oil at 60%, 1.4 g) in small portions while stirring at 0° C. After stirring the reaction mixture for 1 hour, methyl iodide (1.2 mL) was added thereto, and stirred at 20° C. for 20 hrs. After adding ice-cooled water to the reaction fluid, the pH value was adjusted with 1 mol/L hydrochloric acid to become 5. Thus deposited crystals were filtrated, and washed with water. The crystals were dried under a reduced pressure to obtain 2-(1-methylindole-3-yl)-2-oxoacetic acid, methyl ester (1.96 g, 33%).

### Step 3: 2,4-dichlorophenylacetic acid amide

Commercially available 2,4-dichlorophenylacetic acid (12.4 g) was dissolved in methylene chloride (350 mL), and thereto was added oxalyl chloride (10.6 mL) under ice-cooling, followed by stirring at 20° C. for 20 hrs. The reaction fluid was concentrated under a reduced pressure, and the resulting residue was dissolved in methylene chloride (100 mL). This solution was added dropwise to an ice-cooled 28% aqueous ammonia solution (250 mL), and methylene chloride was distilled off under a reduced pressure. Thus deposited crystals were filtrated, and washed with water. The crystals were dried under a reduced pressure to obtain 2,4-dichlorophenylacetic acid amide (11.14 g, 90%).

### Step 4: Synthesis of SB-216763

Tert-butoxy potassium (0.5 g) was dissolved in tetrahydrofuran (35 mL), and thereto were added under ice-cooling the 2-(1-methylindole-3-yl)-2-oxoacetic acid, methyl ester (0.4 g) obtained in the step 2, and then the 2,4-dichlorophenylacetic acid amide (0.3 g) obtained in the step 3, followed by stirring at the same temperature for 3 hrs. After adding water to the reaction fluid, extraction with ethyl acetate was carried out. The organic layer was sequentially washed with a saturated aqueous sodium bicarbonate solution, and saturated salt solution. After drying over anhydrous magnesium sulfate, the crystals were washed with ethanol. The crystals were dried under a reduced pressure to obtain SB-216763 (390 mg, 70%).

¹H-NMR (CDCl₃) δ (ppm): 3.89(s, 3H), 6.41(d, J=8.1 Hz, 1H), 6.80(t, J=7.1 Hz, 1H), 7.15(t, J=7.1 Hz, 1H), 7.32-7.38(m, 3H), 7.49(s, 1H), 8.01(s, 1H), 10.94(s, 1H)

Elementary analysis: theoretical value (C, 61.5; H, 3.3; N, 7.6.), measured value (C, 61.3; H, 3.5; N, 7.4.)

### REFERENCE EXAMPLE 2

### Isolation and Culture of Adult Neural Stem Cell from Rat Brain

After putting a 7-weeks old Sprague Dawley rat to sleep by etherization, decapitation was performed, and the brain was excised through incision of skull from the calvaria. A tissue which includes a part surrounding the cerebral ventricle was isolated from the excised brain under a microscope using scissors and tweezers for ophthalmic use. After dividing the tissue which includes the part surrounding the cerebral ventricle into pieces of approximately 1 mm³ using scissors and scalpel for ophthalmic use, a digesting reaction was allowed in 5 ml of HBSS buffer (manufactured by Invitrogen Corporation) containing 2.5 U/ml papain, 250 U/ml DNase (both manufactured by Worthington, Freehold, N.J.) and 1 U/ml neutral protease (Dispase: manufactured by Boehringer Manheim) at 37° C. for 30 min. A mixture of the cells and tissue obtained following the reaction was washed three times with DMEM (manufactured by Invitrogen Corporation) containing 10% fetal calf serum (manufactured by Hyclone), dissolved in DMEM containing 10% fetal calf serum, and then undigested substances were eliminated using a 10⁷ μm nylon mesh.

Thus resulting crude cell extract was cultured overnight on a 10 cm culture dish using a DMEM/F12 medium (manufactured by Invitrogen Corporation) containing 10% fetal calf serum in an incubator at 37° C. On the following day, the medium was replaced with DMEM/F12 containing a 1% N2 supplement (manufactured by Invitrogen Corporation) and 20 ng/ml FGF-2 (manufactured by PeproTech Inc.) to initiate the culture. Every three days, half of the medium was replaced with fresh DMEM/F12 containing a 1% N2 supplement and 20 ng/ml FGF-2, and the culture was continued. When a small colony of small cells was formed, it was treated with 1% trypsin for approximately from 30 sec to 1 min, and the stripped cells were recovered. Culture of the cells was continued overnight using 10 μg/ml polyornithine (manufactured by Sigma Corporation) at room temperature, and then seeded on a multi-well culture dish (manufactured by Fisher Scientific) which had been coated using 5 μg/ml laminin derived from mouse EHS tumor (manufactured by Becton, Dickinson and Company) at 37° C. overnight, followed by continuing the culture. Through keeping on the aforementioned culture, small thick cells having small sized projections were concentrated. The cells were used in the aforementioned experiment as an adult neural stem cell strain ANSC-7.

### BEST MODE FOR CARRYING OUT THE INVENTION

### Example 1

### Tablet

According to a conventional method, a tablet having the following composition is prepared.

| | | |
|---|---|---|
| (1) | Prescription | |
| | SB-216763 | 5 mg |
| | Lactose | 62 mg |
| | Potato starch | 30 mg |
| | Polyvinyl alcohol | 2 mg |
| | Magnesium stearate | 1 mg |
| | | 100 mg |
| (2) | Kenpaullone | 5 mg |
| | Lactose | 62 mg |
| | Potato starch | 30 mg |
| | Polyvinyl alcohol | 2 mg |
| | Magnesium stearate | 1 mg |
| | | 100 mg |
| (3) | Indirubin-3′-monoxime | 5 mg |
| | Lactose | 62 mg |
| | Potato starch | 30 mg |
| | Polyvinyl alcohol | 2 mg |
| | Magnesium stearate | 1 mg |
| | | 100 mg |

### Example 2

### Agent for the Promotion of Neuropoiesis of a Neural Stem Cell (1)

According to a conventional method, lithium chloride was dissolved in PBS to give 3 mol/l to prepare an agent for the promotion of neuropoiesis of a neural stem cell containing lithium chloride.

### Example 3

### Agent for the Promotion of Neuropoiesis of a Neural Stem Cell (2)

According to a conventional method, SB-216763, Kenpaullone or indirubin-3′-monoxime was dissolved in DMSO to give 0.1 mmol/l to prepare an agent for the promotion of neuropoiesis of a neural stem cell containing SB-216763, Kenpaullone or indirubin-3′-monoxime.

### INDUSTRIAL APPLICABILITY

According to the present invention, a nerve regenerating drug comprising a substance that inhibits the activity of glycogen synthase kinase-3, as an active ingredient; an agent for the promotion of neuropoiesis of a neural stem cell comprising the substance as an active ingredient; a neuron obtained by culturing a neural stem cell in the presence of the agent for the promotion of neuropoiesis; and a method of the manufacture of the neuron can be provided.

Free Text of the Sequence Listing

SEQ ID. NO: 5—Description of artificial sequence: synthetic protein

SEQ ID NO: 6—Description of artificial sequence: synthetic DNA

SEQ ID NO: 7—Description of artificial sequence: synthetic DNA

SEQ. ID NO: 8—Description of artificial sequence: synthetic DNA

SEQ ID NO: 9—Description of artificial sequence: synthetic DNA

SEQ ID NO: 10—Description of artificial sequence: synthetic DNA

SEQ ID NO: 11—Description of artificial sequence: synthetic DNA

SEQ ID NO: 12—Description of artificial sequence: synthetic DNA

SEQ ID NO: 13—Description of artificial sequence: synthetic DNA

SEQ ID NO: 14—Description of artificial sequence: synthetic RNA

SEQ ID NO: 15—Description of artificial sequence: synthetic RNA

SEQ ID NO: 16—Description of artificial sequence: synthetic RNA

SEQ ID NO: 17—Description of artificial sequence: synthetic RNA

1. A method for regenerating nerve comprising administering to a patient in need thereof, a therapeutically effective amount of a pharmaceutical composition which comprises a substance that inhibits the activity of GSK-3, as an active ingredient.

2. The method according to claim 1 wherein the pharmaceutical composition is a therapeutic drug for a neurological disease.

3. The method according to claim 2 wherein the neurological disease is selected from the group consisting of Parkinson's disease, Alzheimer's disease, Down's disease, cerebrovascular disorder, cerebral stroke, spinal cord injury, Huntington's chorea, multiple sclerosis, amyotrophic lateral sclerosis, epilepsy, anxiety disorder, schizophrenia, depression and manic depressive psychosis.

4. The method according to any one of claims 1 to 3 wherein the substance that inhibits the activity of GSK-3 is lithium or a pharmacologically acceptable salt thereof.

5. The method according to any one of claims 1 to 3 wherein the substance that inhibits the activity of GSK-3 comprises a bisindolylmaleimide derivative, a 3-aryl-4-indolylmaleimide derivative, an indolocarbazole derivative, an indolo[3,2-d][1]benzazepin-6(5H)-one derivative or an indirubin derivative, or a pharmacologically acceptable salt thereof.

6. The method according to any one of claims 1 to 3 wherein the substance that inhibits the activity of GSK-3 comprises a compound represented by the formula (I): 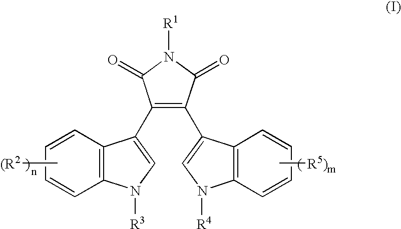
[wherein n and m may be the same or different, and represent an integer of 1 to 3; R¹, R³ and R⁴ may be the same or different, and represent hydrogen, substituted or unsubstituted lower alkyl, substituted or unsubstituted lower alkenyl, —COR⁶ (wherein R⁶ represents hydrogen, substituted or unsubstituted lower alkyl, substituted or unsubstituted lower alkenyl, substituted or unsubstituted aryl or substituted or unsubstituted cycloalkyl), —COOR⁷ (wherein R⁷ represents hydrogen, substituted or unsubstituted lower alkyl, substituted or unsubstituted aryl or substituted or unsubstituted cycloalkyl) or —OR⁸ (wherein R⁸ represents hydrogen, substituted or unsubstituted lower alkyl, substituted or unsubstituted aryl or substituted or unsubstituted cycloalkyl); R² and R⁵ may be the same or different, and represent hydrogen, substituted or unsubstituted lower alkyl, a substituted or unsubstituted lower alkenyl, substituted or unsubstituted lower alkoxy, substituted or unsubstituted lower alkoxycarbonyl, substituted or unsubstituted aryl, carboxy, halogen, hydroxy, nitro, amino, or mono- or di-lower alkylamino; when n and m are 2 or 3, each of R² and R⁵ may be the same or different],
a compound represented by the formula (II): 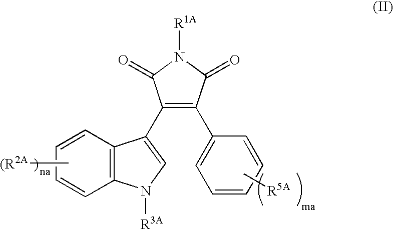
(wherein na, ma, R^{1A}, R^{2A}, R^{3A} and R^{5A} are as defined for the aforementioned n, m, R¹, R², R³ and R⁵, respectively, or
a compound represented by the formula (III): 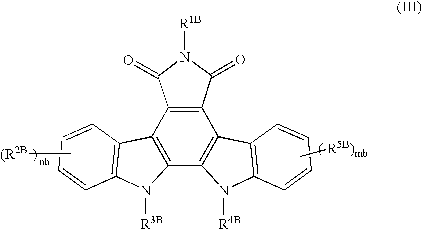
[wherein nb, mb, R^{1B}, R^{2B} and R^{5B} are as defined for the aforementioned n, m, R¹, R² and R⁵, respectively; R^{3B} and R^{4B} may be the same or different, and represent hydrogen, substituted or unsubstituted lower alkyl, substituted or unsubstituted lower alkenyl, —COR⁶, —COOR⁷ or —OR⁸, or R^{3B} and R^{4B} together form 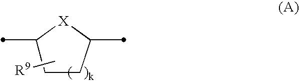
(wherein k represents 1 or 2; X represents CH₂, NH, an oxygen atom or a sulfur atom; R⁹ represents hydroxy, carboxy, carbamoyl or lower alkoxycarbonyl)];
or a pharmacologically acceptable salt thereof.

7. The method according to any one of claims 1 to 3 wherein the substance that inhibits the activity of GSK-3 is a compound represented by the formula (Ia): 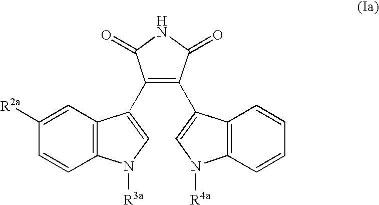
(wherein R^{2a} represents hydrogen, lower alkoxy, lower alkoxycarbonyl, aryl or nitro; R^{3a} and R^{4a} may be the same or different, and represent substituted or unsubstituted lower alkyl),
or a pharmacologically acceptable salt thereof.

8. The method according to any one of claims 1 to 3 wherein the substance that inhibits the activity of GSK-3 is a compound represented by the formula (IIa): 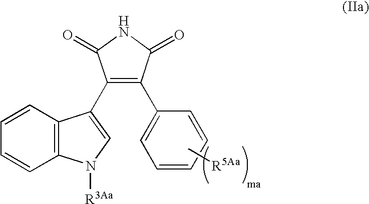
(wherein R^{3Aa} represents substituted or unsubstituted lower alkyl; R^{5Aa} represents halogen),
or a pharmacologically acceptable salt thereof.

9. The method according to any one of claims 1 to 3 wherein the substance that inhibits the activity of GSK-3 is a compound represented by the formula (IIIa): 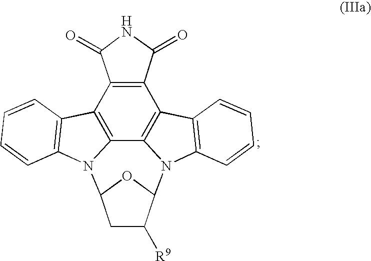
or a pharmacologically acceptable salt thereof.

10. The method according to any one of claims 1 to 3 wherein the substance that inhibits the activity of GSK-3 is a compound selected from the group consisting of 3,4-bis(1-methylindole-3-yl)-1H-pyrrole-2,5-dione, 3-(1-methylindole-3-yl)-4-(1-propylindole-3-yl)-1H-pyrrole-2,5-dione, 3-[1-(3-cyanopropyl)indole-3-yl]-4-(1-methylindole-3-yl)-1H-pyrrole-2,5-dione, 3-[1-(3-aminopropyl)indole-3-yl]-4-(1-methylindole-3-yl)-1H-pyrrole-2,5-dione, 3-[1-(3-carboxypropyl)indole-3-yl]-4-(1-methylindole-3-yl)-1H-pyrrole-2,5-dione, 3-[1-(3-carbamoylpropyl)indole-3-yl]-4-(1-methylindole-3-yl)-1H-pyrrole-2,5-dione, 3-[1-(3-aminopropyl)indole-3-yl]-4-(1-methyl-5-propyloxyindole-3-yl)-1H-pyrrole-2,5-dione, 3-[1-(3-hydroxypropyl)indole-3-yl]-4-(1-methyl-5-phenylindole-3-yl)-1H-pyrrole-2,5-dione, 3-[1-(3-aminopropyl)indole-3-yl]-4-(1-methyl-5-phenylindole-3-yl)-1H-pyrrole-2,5-dione, 3-[1-(3-hydroxypropyl)indole-3-yl]-4-(1-methyl-5-methoxycarbonylindole-3-yl)-1H-pyrrole-2,5-dione, 3-[1-(3-hydroxypropyl)indole-3-yl]-4-(1-methyl-5-nitroindole-3-yl)-1H-pyrrole-2,5-dione, 3-(1-methylindole-3-yl)-4-[1-(3-hydroxypropyl)-5-nitroindole-3-yl]-1H-pyrrole-2,5-dione, 3-(2-chlorophenyl)-4-(1-methylindole-3-yl)-1H-pyrrole-2,5-dione, 3-(2,4-dichlorophenyl)-4-(1-methylindole-3-yl)-1H-pyrrole-2,5-dione, 3-(2-chlorophenyl)-4-[1-(3-hydroxypropyl)indole-3-yl]-1H-pyrrole-2,5-dione, 4-[1-(3-aminopropyl)indole-3-yl]-3-(2-chlorophenyl)-1H-pyrrole-2,5-dione and 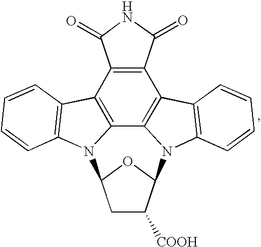
or a pharmacologically acceptable salt thereof.

11. The method according to any one of claims 1 to 3 wherein the substance that inhibits the activity of GSK-3 comprises a compound represented by the formula (IV): 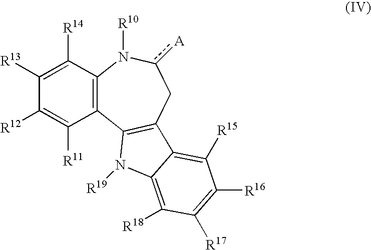
[wherein A is oxygen or sulfur coupled to the right by a single or double bond; R¹⁰ is selected from the group consisting of hydrogen, aryl, lower aliphatic substituents, particularly alkyl and lower alkyl ester; R¹¹-R¹⁴ are independently selected from the group consisting of alkoxy, amino, acyl, aliphatic substituents, particularly alkyl, alkenyl and alkinyl substituents, aliphatic alcohols, particularly alkyl alcohols, aliphatic nitriles, particularly alkyl nitriles, cyano, nitro, carboxyl, halogen, hydrogen, hydroxyl, imino and α,β-unsaturated ketones; R¹⁵-R¹⁸ are independently selected from the group consisting of aliphatic substituents, particularly alkyl, alkenyl and alkinyl substituents, particularly lower aliphatic substituents, alipahatic alcohols, particularly alkyl alcohols, alkoxy, acyl, cyano, nitro, epoxy, haloalkyl groups, halogen, hydrogen and hydroxyl; R¹⁹ is selected from the group consisting of aliphatic groups, particularly lower alkyl groups, aliphatic alcohols, particularly alkyl alcohols, carboxylic acids and hydrogen],
or a pharmacologically acceptable salt thereof.

12. The method according to any one of claims 1 to 3 wherein the substance that inhibits the activity of GSK-3 is a compound selected from the group consisting of 7,12-dihydro-indolo[3,2-d][1]benzazepin-6(5H)-one, 2-bromo-7,12-dihydro-indolo[3,2-d][1]benzazepin-6(5H)-one, 9-bromo-7,12-dihydro-indolo[3,2-d][1]benzazepin-6(5H)-one, 9-chloro-7,12-dihydro-indolo[3,2-d][1]benzazepin-6(5H)-one, 11-chloro-7,12-dihydro-indolo[3,2-d][1]benzazepin-6(5H)-one, 10-bromo-7,12-dihydro-indolo[3,2-d][1]benzazepin-6(5H)-one, 8-bromo-6,1-dihydro-thieno[3′,2′:2,3]azepino[4,5-b]indol-5(4H)-one, 9-bromo-7,12-dihydro-4-methoxy-indolo[3,2-d][1]benzazepin-6(5H)-one, 9-bromo-7,12-dihydro-4-hydroxy-indolo[3,2-d][1]benzazepin-6(5H)-one, 7,12-dihydro-4-methoxy-indolo[3,2-d][1]benzazepin-6(5H)-one, 9-bromo-7,12-dihydro-2,3-dimethoxy-indolo[3,2-d][1]benzazepin-6(5H)-one, 9-bromo-7,12-dihydro-2,3-dihydroxy-indolo[3,2-d][1]benzazepin-6(5H)-one, 7,12-dihydro-2,3-dimethoxy-indolo[3,2-d][1]benzazepin-6(5H)-one, 7,12-dihydro-9-trifluormethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2-bromo-7,12-dihydro-9-trifluoromethyl-indolo[3,2-d][1]benzazepin-6(5H)-one, 9-bromo-7,12-dihydro-indolo[3,2-d][1]benzazepin-6(5H)-thione, 9-bromo-5,12-bis-(t-butyloxycarbonyl)-7,12-dihydro-indolo[3,2-d][1]-benzazepin-6(5H)-one, 9-bromo-12-(t-butyloxycarbonyl)-7,12-dihydro-indolo[3,2-d][1]benzazepin-6(5H)-one, 9-bromo-5,7-bis-(t-butyloxycarbonyl)-7,12-dihydro-indolo-[3,2-d][1]benzazepin-6(5H)-one, 9-bromo-5,7,12-tri-(t-butyloxycarbonyl)-7,12-dihydro-indolo[3,2-d][1]benzazepin-6(5H)-one, 9-bromo-7,12-dihydro-5-methyloxycarbonylmethyl-indolo[3,2-d][1]benzazepin-6(5H)-one, 9-bromo-7,12-dihydro-12-methyloxycarbonylmethyl-indolo[3,2-d][1]benzazepin-6(5H)-one, 9-bromo-7,12-dihydro-12-(2-hydroxyethyl)-indolo[3,2-d][1]benzazepin-6(5H)-one, 2,9-dibromo-7,12-dihydro-indolo[3,2-d][1]benzazepin-6(5H)-one, 8,10-dichloro-7,12-dihydro-indolo[3,2-d][1]benzazepin-6(5H)-one, 9-cyano-7,12-dihydro-indolo[3,2-d][1]benzazepin-6(5H)-one, 9-bromo-7,12-dihydro-5-methyl-indolo[3,2-d][1]benzazepin-6(5H)-one, 5-benzyl-9-bromo-7,12-dihydro-5-methyl-indolo[3,2-d][1]benzazepin-6(5H)-one, 9-bromo-7,12-dihydro-12-methyl-indolo[3,2-d][1]benzazepin-6(5H)-one, 9-bromo-12-ethyl-7,12-dihydro-indolo[3,2-d][1]benzazepin-6(5H)-one, 9-bromo-7,12-dihydro-12-(2-propenyl)-indolo[3,2-d][1]benzazepin-6(5H)-one, 7,12-dihydro-9-methyl-indolo[3,2-d][1]-benzazepin-6(5H)-one, 7,12-dihydro-9-methoxy-indolo[3,2-d][1]benzazepin-6(5H)-one, 9-fluoro-7,12-dihydro-12-(2-propenyl)-indolo[3,2-d][1]benzazepin-6(5H)-one, 11-bromo-7,12-dihydro-indolo[3,2-d][1]benzazepin-6(5H)-one, 9-bromo-7,12-dihydro-2-(methyliminoamine)-indolo[3,2-d][1]benzazepin-6(5H)-one, 9-bromo-7,12-dihydro-2-(carboxylic acid)-indolo[3,2-d][1]benzazepin-6(5H)-one, 9-bromo-7,12-dihydro-10-hydroxy-indolo[3,2-d][1]benzazepin-6(5H)-one, 9-bromo-7,12-dihydro-11-hydroxymethyl-indolo[3,2-d][1]benzazepin-6(5H)-one, 7,12-dihydro-4-hydroxy-indolo[3,2-d][1]benzazepin-6(5H)-one, 7,12-dihydro-2,3-dihydroxy-indolo[3,2-d][1]benzazepin-6(5H)-one, 2,3-dimethoxy-9-nitro-7,12-dihydro-indolo[3,2-d][1]benzazepin-6(5H)-one, 9-cyano-7,12-dihydro-indolo[3,2-d][1]benzazepin-6(5H)-one, 2,3-dimethoxy-9-cyano-7,12-dihydro-indolo[3,2-d][1]benzazepin-6(5H)-one, 9-nitro-7,12-dihydro-indolo[3,2-d][1]benzazepin-6(5H)-one, 3-(6-oxo-9-trifluoromethyl-5,6,7,12-tetrahydro-indolo[3,2-d][1]benzazepin-2-yl)-propionitrile, 2-bromo-9-nitro-7,12-dihydro-indolo[3,2-d][1]benzazepin-6(5H)-one, 3-(6-oxo-9-trifluoromethyl-5,6,7,12-tetrahydro-indolo[3,2-d][1]benzazepin-2-yl)acrylonitrile, 2-(3-hydroxy-1-propinyl)-9-trifluoromethyl-7,12-dihydro-indolo[3,2-d][1]benzazepin-6(5H)-one, 2-iodo-9-bromo-7,12-dihydro-indolo[3,2-d][1]benzazepin-6(5H)-one, 2-(3-oxo-1-butenyl)-9-trifluoromethyl-7,12-tetrahydro-indolo[3,2-d][1]benzazepin-6(5H)-one, 8-chloro-6,1-dihydro-thieno[3′,2′:2,3]azepino[4,5-b]indol-5(4H)-one, 2-iodo-9-trifluoromethyl-7,12-dihydro-indolo[3,2-d][1]benzazepin-6(5H)-one, 7,12-dihydro-pyrido[3′,2′:4,5]pyrrolo[3,2-d][1]benzazepin-6(5H)-one, 11-methyl-7,12-dihydro-indolo[3,2-d][1]-benzazepin-6(5H)-one, 2-[2-(1-hydroxycyclohexyl)ethinyl]-9-trifluoromethyl-7,12-dihydro-indolo[3,2-d][1]benzazepin-6(5H)-one, 2-cyano-7,12-dihydro-indolo[3,2-d][1]benzazepin-6(5H)-one, 2-iodo-7,12-dihydro-indolo[3,2-d][1]benzazepin-6(5H)-one, 11-ethyl-7,12-dihydro-indolo[3,2-d][1]benzazepin-6(5H)-one, 8-methyl-6,11-dihydro-thieno[3′,2′:2,3]azepino[4,5-b]indol-5(4H)-one and 3-(6-oxo-9-trifluoromethyl-5,6,7,12-tetrahydro-indolo[3,2-d][1]benzazepin-2-yl)acrylic acid, methyl ester, or a pharmacologically acceptable salt thereof.

13. The method according to any one of claims 1 to 3 wherein the substance that inhibits the activity of GSK-3 is selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]benzazepin-6(5H)-one, 7,12-dihydro-9-trifluoromethyl-indolo[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indole-5(4H)-one, 7,12-dihydro-9-methoxy-indolo[3,2-d][1]benzazepin-6(5H)-one, 10-bromo-7,12-dihydro-indolo[3,2-d][1]benzazepin-6(5H)-one, 11-bromo-7,12-dihydro-indolo[3,2-d][1]benzazepin-6(5H)-one, 11-chloro-7,12-dihydro-indolo[3,2-d][1]benzazepin-6(5H)-one, 9-fluoro-7,12-dihydro-indolo[3,2-d][1]benzazepin-6(5H)-one, 9-methyl-7,12-dihydro-indolo[3,2-d][1]-benzazepin-6(5H)-one, 9-bromo-7,12-dihydro-indolo[3,2-d][1]benzazepin-6(5H)-thione, 8,10-dichloro-7,12-dihydro-indolo[3,2-d][1]benzazepin-6(5H)-one, 9-bromo-7,12-dihydro-12-(2-hydroxyethyl)-indolo[3,2-d][1]benzazepin-6(5H)-one, 9-bromo-7,12-dihydro-2,3-dihydroxy-indolo[3,2-d][1]benzazepin-6(5H)-one, 2-bromo-7,12-dihydro-indolo[3,2-d]-[1]benzazepin-6(5H)-one, 7,12-dihydro-2,3-dimethoxy-indolo[3,2-d][1]benzazepin-6(5H)-one, 9-bromo-7,12-dihydro-12-methyl-indolo[3,2-d][1]benzazepin-6(5H)-one, 9-bromo-7,12-dihydro-5-methyloxycarbonylmethyl-indolo[3,2-d][1]benzazepin-6(5H)-one and 7,12-dihydro-indolo[3,2-d][1]benzazepin-6(5H)-one,
or a pharmacologically acceptable salt thereof.

14. The method according to any one of claims 1 to 3 wherein the substance that inhibits the activity of GSK-3 is selected from the group consisting of 9-cyano-7,12-dihydro-indolo[3,2-d][1]benzazepin-6(5H)-one, 9-bromo-7,12-dihydro-2,3-dimethoxy-indolo[3,2-d][1]benzazepin-6(5H)-one, 2-bromo-7,12-dihydro-9-trifluoromethyl-indolo[3,2-d][1]benzazepin-6(5H)-one, 7,12-dihydro-2,3-dimethoxy-9-trifluoromethyl-indolo[3,2-d][1]benzazepin-6(5H)-one, 2,9-dibromo-7,12-dihydro-indolo[3,2-d][1]benzazepin-6(5H)-one, 7,12-dihydro-9-trifluormethyl-indolo[3,2-d][1]benzazepin-6(5H)-one, 9-chloro-7,12-dihydro-indolo[3,2-d][1]benzazepin-6(5H)-one, 8-bromo-6,11-dihydro-thieno[3′,2′:2,3]azepino[4,5-b]indol-5(4H)-one, 7,12-dihydro-9-methoxy-indolo[3,2-d][1]benzazepin-6(5H)-one,
or a pharmacologically acceptable salt thereof.

15. The method according to any one of claims 1 to 3 wherein the substance that inhibits the activity of GSK-3 is 9-bromo-7,12-dihydro-indolo[3,2-d][1]benzazepin-6(5H)-one, or a pharmacologically acceptable salt thereof.

16. The method according to any one of claims 1 to 3 wherein the substance that inhibits the activity of GSK-3 comprises a compound represented by the formula (V): 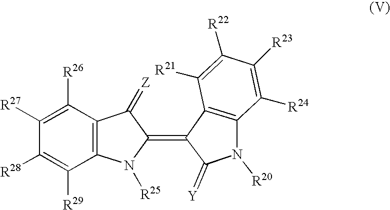
[wherein R²⁰ and R²⁵ may be the same or different and represent hydrogen; halogen; a hydroxy group; a methylene hydroxy group; a straight or branched C₁ to C₁₈-alkyl or straight or branched C₁ to C₁₈-alkoxy or a methylenealkoxy group (wherein the alkoxy is straight or branched C₁ to C₁₈); a cycloalkyl group having 3 to 7 carbon which may have one or more heteroatoms a substituted or unsubstituted aryl, aralkyl or aryloxy group which may have one or more heteroatoms; a mono-, di- or trialkylsilyl group each independently having 1 to 6 carbon atoms within the straight or branched alkyl group; a mono-, di- or triarylsilyl group each independently having a substituted or unsubstituted aryl group; a trifluoromethyl group; —COM; —COOM; or a —CH₂COOM group (wherein M represents hydrogen, a straight or branched C₁ to C₁₈-alkyl group which may be substituted with one or more hydroxy and/or amino groups, or an aryl group, which may be substituted with one or more halogen, alkyl groups or alkoxy groups which may have one or more heteroatoms; an —NR³⁰R³¹ group (wherein R³⁰ and R³¹ may be the same or different and represent a hydrogen atom, a C₁ to C₁₈ straight or branched alkyl group which may be additionally substituted with one or more hydroxy and/or amino groups, a substituted or unsubstituted aryl group which may have one or more heteroatoms); an acyl group; a —CH₂—NR³⁰R³¹ methyleneamino group; a benzyl group which may have one or more heteroatoms in the benzene ring; a methylenecycloalkyl group having 3 to 7 carbon atoms which may have one or more heteroatoms; a physiological amino acid group coupled to a nitrogen atom as an amide; an O-glycoside or N-glycoside having glycoside of which being selected from monosaccharides or disaccharides; or a methylenesulfonate group; R²¹R²², R²³, R²⁴, R²⁶, R²⁷, R²⁸ and R²⁹ may be the same or different and represent hydrogen; halogen; a hydroxy group; a nitroso group; a nitro group; an alkoxy group; a straight or branched C₁ to C₁₈ alkyl group which may be substituted with one or more hydroxy and/or amino groups; a substituted or unsubstituted aryl group which may have one or more heteroatoms; a substituted or unsubstituted aralkyl group which may have one or more heteroatoms; a substituted or unsubstituted aryloxy group which may have one or more heteroatoms; a substituted or unsubstituted methylenearyloxy group which may have one or more heteroatoms; a cycloalkyl group having 3 to 7 carbon atoms which may have one or more heteroatoms; a methylenecycloalkyl group having 3 to 7 carbon atoms which may have one or more heteroatoms; a trifluoromethyl group; —COM; —COOM; or a CH₂COOM group (wherein M represents hydrogen, a straight or branched C₁ to C₁₈-alkyl group which may be additionally substituted with one or more hydroxy and/or amino groups, or an aryl group, which may be substituted with one or more halogen atoms, alkyl groups or alkoxy groups which may have one or more heteroatoms); an —NR³⁰R³¹ group (wherein R³⁰ and R³¹ which may be the same or different and represent hydrogen, a straight or branched C₁ to C₁₈-alkyl group which may be additionally substituted with one or more hydroxy and/or amino groups, a substituted or unsubstituted aryl group which may have one or more heteroatoms, an acyl group, or form a part of cycloalkyl having 3 to 7 carbon atoms with the nitrogen atom which may have one or more heteroatoms); a —CONR³⁰R³¹ group; a hydroxylamino group; a phosphate group; a phosphonate group; a sulfate group; a sulfonate group; a sulfonamide group; an —SO₂NR³⁰R³¹ group; an —N═N—R³² azo group (wherein R³² represents an aromatic group which may be substituted with one or more carboxyl, phosphoryl or sulfonate groups, or an O-glycoside or N-glycoside group having glycoside of which being selected from monosaccharides or disaccharides); or R²⁰ and R²⁴, and R²⁵ and R²⁹ together form a ring which may have one to four CH₂ groups each independently substituted, respectively; Y and Z may be the same or different and represent an oxygen atom; a sulfur atom; a selenium atom; a tellurium atom; an NR³³ group (wherein R³³ represents hydrogen, a straight or branched C₁ to C₁₈ alkyl group which may be substituted with one or more carboxyl, phosphoryl or sulfonate groups, a substituted or unsubstituted aryl group which may have one or more heteroatoms, an aralkyl group or a sulfonate group); or —NOR³³],
or a pharmacologically acceptable salt thereof.

17. The method according to any one of claims 1 to 3 wherein the substance that inhibits the activity of GSK-3 is a compound selected from the group consisting of indirubin, 5-iodo-indirubin, 5-bromo-indirubin, 5-chloro-indirubin, 5-fluoro-indirubin, 5-methyl-indirubin, 5-nitro-indirubin, 5-SO₃H-indirubin, 5′-bromo-indirubin, 5-5′-dibromo-indirubin and 5′-bromo-indirubin 5-sulfonic acid,
or a pharmacologically acceptable salt thereof.

18. The method according to any one of claims 1 to 3 wherein the substance that inhibits the activity of GSK-3 is a compound selected from the group consisting of indirubin-3′-monooxime, 5-iodo-indirubin-3′-monooxime and 5-SO₃Na-indirubin-3′-monooxime,
or a pharmacologically acceptable salt thereof.

19. The method according to any one of claims 1 to 3 wherein the substance that inhibits the activity of GSK-3 is indirubin-3′-monooxime or a pharmacologically acceptable salt thereof.

20-37. (canceled)

38. A method of the manufacture of a neuron which comprises culturing a neural stem cell in the presence of the substance that inhibits the activity of GSK-3 to allow neogenesis of the neuron, and collecting the neuron from the culture.

39-41. (canceled)
